(19) United States
(12) Patent Application Publication
(10) Pub. No.: US 2025/0381071 A1
(43) Pub. Date: Dec. 18, 2025

(54) REUSABLE MOISTURE RETAINING ARTICLE

(71) Applicant: RSD HOLDINGS LIMITED, AUCKLAND (NZ)

(72) Inventor: FRANTISEK RIHA-SCOTT, AUCKLAND (NZ)

(73) Assignee: RSD HOLDINGS LIMITED, AUCKLAND (NZ)

(21) Appl. No.: 18/881,029

(22) PCT Filed: Jul. 4, 2023
(86) PCT No.: PCT/IB2023/056914

§ 371 Date: Jan. 3, 2025

(30) Foreign Application Priority Data
Jul. 4, 2022 (AU) 2022901875

(51) Int. Cl. A61F 13/15; A61F 13/471; A61F 13/472; A61F 13/505; A61F 13/539

(52) U.S. Cl. CPC A61F 13/15268; A61F 13/15731; A61F 13/15756; A61F 13/471; A61F 13/472; A61F 13/505; A61F 13/539; A61F 2013/53908

(57) ABSTRACT

Disclosed is a reusable moisture retaining article 100 of or for use with an undergarment to be positioned adjacent a body of a user and to absorb and retain moisture from the user. The article comprises a reusable moisture absorbing and retaining insert 110 at least a portion of which is exposed or configured to be exposed to define an inner layer of the article that is configured to be in direct contact with the user's body to absorb moisture. An outer cover 120 defines an outer layer of the article. An adhesive layer 130 interconnects the insert 110 and the outer cover 120. The insert 110 and the outer cover 120 are bonded together with the adhesive layer 110 by applying a compressive force.

### FIELD OF INVENTION

The present invention relates to a reusable moisture retaining article, and in particular but not exclusively to a reusable urine incontinence retaining article or a reusable menstrual fluid retaining article that may form part of or be used with undergarments to be worn by a person.

### BACKGROUND

Disposable or single use absorbent articles are commercially available in a wide variety of configurations for absorbing body exudates or moistures. Single use urine incontinence absorbent articles or single use menstrual fluid absorbent articles are used to absorb body exudates or moistures from pudendal region of the wearer. However, such single disposable articles are not intended to be reusable and therefore needs to be disposed after use. Such disposable articles are also not environmentally friendly and safe disposal of such articles can be a problem.

Current market available reusable absorbent products (e.g., reusable menstrual and urine incontinence products) are very complex to manufacture due to the performance needs requiring many manual labour steps, thereby making them a very expensive option for consumers in comparison to disposable alternatives in the early stages. Furthermore, currently available reusable products perform below any disposable alternative and leak.

There is a need for reusable moisture retaining articles that are cost effective to manufacture without compromising on the performance.

### OBJECT OF THE INVENTION

It is therefore an object of the present invention to provide a reusable body moisture retaining article which overcomes or at least ameliorates some of the disadvantages described above, or which at least provides a public with a useful choice.

### BRIEF DESCRIPTION OF THE INVENTION

In a first aspect, the present invention resides in a method of constructing a reusable moisture retaining article of or for use with an undergarment to be positioned adjacent a body of a user and to absorb and retain a moisture that is a body moisture from the user, the method comprising:
providing a reusable moisture absorbing and retaining insert at least a portion of which is exposed or configured to be exposed to define an inner layer of the article, the inner layer being configured to be located adjacent the user's body and facing towards the user's body for being in bodily contact with the user to directly absorb and retain the body moisture from the user;
providing an outer cover that is configured to define an outer layer of the article;
positioning an adhesive layer or at least a portion of an/the adhesive layer between the insert and the outer cover between the insert and the outer cover interconnecting the insert and the outer cover; and
applying a compressive force thereby bonding the insert and the outer cover together by the adhesive layer.

Preferably, the method further comprises:
providing an inner more layer;
providing a protective layer that is waterproof;
providing a further adhesive layer that is a second adhesive layer;
positioning the protective layer on a surface of the insert that is configured to face towards the user's body;
positioning the second adhesive layer so that at least a portion of the second adhesive layer is located between the protective layer and the inner more layer for interconnecting at least the protective layer and the inner more layer;
applying a further compressive force to at least the insert and the at least one inner more layer; and
applying a heat and adhesive bonding thereby bonding the protective layer with the inner more layer and the insert.

Preferably, the compressive force is applied to the article.

Preferably, the compressive force is applied to the insert and the outer cover.

Preferably, the method comprises applying a compressive force to at least the insert and the outer cover.

Preferably, the method comprises applying heat and adhesive bonding to bond the insert and the outer cover together using the first adhesive layer.

Preferably, the heat bonding is applied at temperature between 120 to 200 degrees Celsius.

Preferably, the method comprises positioning at least a further portion of the second adhesive layer between the insert and the inner more layer for interconnecting the insert and the inner more layer to bond the insert with inner more layer using the second adhesive layer.

Preferably, the protective layer is formed as at least one protective strip.

Preferably, the protective layer is formed as at least two protective strips.

Preferably, the protective layer is a waterproof layer.

Preferably, the method comprising positioning or sandwiching the protective strip between the insert and the second adhesive layer on the surface of insert that is configured to face towards the user's body.

Preferably, the method comprises positioning or sandwiching the at least two protective strips between the insert and the second adhesive layer on the surface of insert that is configured to face towards the user's body and along at least two edges of the insert.

Preferably, the protective layer is formed as a single piece frame.

Preferably, the method comprises positioning or sandwiching the at least two protective strips between the insert and the second adhesive layer on the surface of insert that is configured to face towards the user's body and along at least two edges of the insert.

Preferably, the method comprises bonding the insert and the outer cover together only by the adhesive layer.

Preferably, the method comprises bonding the insert and the outer cover together only by adhesive and heat bonding.

Preferably, the method further comprises:
providing an or the inner more layer located outwardly of the insert;
providing a second adhesive layer and positioning the insert and the inner more layer between the inner more layer and the insert;
applying compressive force and bonding (preferably adhesive and heat bonding) the insert with the inner more layer by applying a compressive force.

Preferably, the method comprises:
providing the protective layer (or at least one protective strip) that is waterproof;
positioning the protective layer (or the at least one protective strip) between the insert and the second adhesive layer on the surface of insert that is configured to face towards the user's body;
providing a further adhesive layer (third adhesive layer) and positioning it between the protective layer (or the protective strip(s)) and the insert thereby interconnecting the protective layer (or protective strip(s)) and the insert; and
applying compressive force and bonding the protective layer (or the at least one protective strip) with at least the insert by applying a compressive force.

Preferably, the insert and the protective layer are bonded together only by the further adhesive layer (third adhesive layer).

Preferably, the method comprises bonding the insert and the at least one adhesive layer together only by adhesive and heat bonding.

Preferably, the method comprises:
providing the protective layer (or at least one protective strip) that is waterproof;
positioning the protective layer (or the at least one protective strip) between the insert and the second adhesive layer on the surface of insert that is configured to face towards the user's body;
applying compressive force and bonding (preferably by adhesive bonding or by adhesive and heat bonding) the protective layer (or the at least one protective strip) with at least the insert.

Preferably, the article is configured to be positioned adjacent a pudendal region of the user and to absorb and retain the moisture from the pudendal region of the user.

Preferably, the moisture is or comprises menstrual fluid and/or urine incontinence.

Preferably, the adhesive layer is coextensive with the insert.

Preferably, the adhesive layer is coextensive with the outer cover.

Preferably, the adhesive layer is coextensive with the insert and the outer cover.

Preferably, the adhesive layer is or comprises a bonding glue.

Preferably, the adhesive layer is non-woven.

Preferably, the adhesive layer is fused with waterproof textile.

Preferably, the outer cover is the outer most layer of the article.

Preferably, the outer cover is a decorative layer of the article.

Preferably, the outer cover is a protective layer to the insert and/or to the adhesive layer.

Preferably, the outer cover comprises or is made from woven and/or knitted textiles.

Preferably, the outer cover is coextensive with the insert.

Preferably, the outer cover is moisture resistant.

Preferably, a moisture resistant solution is applied to the outer cover.

Preferably, the outer cover is coated with a moisture resistant solution.

Preferably, the outer cover is a leakproof layer.

Preferably, the outer cover extends from the top end of the article to the bottom end of the article.

Preferably, the outer cover comprises one or more anti-slip prints at its outermost surface.

Preferably, the one or more anti-slip prints are rubber prints.

Preferably, the anti-slip prints are made out of a silicon material.

Preferably, the silicon material glows in the dark.

Preferably, the insert is made from woven and/or knitted textile(s).

Preferably, the article further comprises an or the inner more layer located outwardly of the insert and bonded (preferably by adhesive bonding or by adhesive and heat bonding) to the insert using a second adhesive layer, the second adhesive layer being positioned or sandwiched between the insert and the inner more layer bonding (preferably by adhesive bonding or by adhesive and heat bonding) the inner more layer and the insert.

Preferably, the second adhesive layer is an adhesive strip.

Preferably, the second adhesive layer is or comprises a bonding glue.

Preferably, the second adhesive layer is non-woven.

Preferably, the inner more layer is the inner most layer of the article.

Preferably, the inner more layer comprises or is made from woven or knitted textile(s).

Preferably, the inner more layer is moisture resistant.

Preferably, a moisture resistant solution is applied to the inner more layer.

Preferably, the inner more layer is coated with a moisture resistant solution.

Preferably, the inner more layer defines a window to allow the portion of insert to be exposed or configured to be exposed.

Preferably, the inner more layer comprises or is in the form of a first inner more strip and a second inner more strip and the second adhesive layer comprises or is in the form of a first adhesive strip and a second adhesive strip, wherein the first inner more strip is located outwardly of the insert and bonded (preferably adhesive bonded or adhesive and heat bonded) to the insert using the first adhesive strip and the second inner more strip is located outwardly of the insert and is bonded (preferably adhesive bonded or adhesive and heat bonded) to the insert using the second adhesive strip, the first adhesive strip being positioned or sandwiched between the insert and the first inner more strip bonding (preferably adhesive bonding or adhesive and head bonding) the first inner more strip and the insert, and the second adhesive strip being positioned or sandwiched between the insert and the second inner more strip bonding (preferably adhesive bonding or adhesive and head bonding) the second inner more strip and the insert.

Preferably, substantial portion of the insert is exposed or configured to be exposed to form the inner layer of the article.

Preferably, all of the insert is exposed or configured to be exposed to form the inner layer of the article save for its edges.

Preferably, the article comprises at least one protective strip that is waterproof that is positioned or sandwiched between the insert and the second adhesive layer on the surface of insert that is configured to face towards the user's body.

Preferably, the article comprises at least two protective strips that are waterproof and are positioned or sandwiched between the insert and the second adhesive layer on the surface of insert that is configured to face towards the user's body and along at least two edges of the insert.

Preferably, the protective strip(s) are positioned or sandwiched between the insert and the second adhesive layer on the surface of insert that is configured to face towards the user's body and along all edges/perimeter of the insert.

Preferably, protective strips are formed as a single piece frame.

Preferably, the or each protective strip is bonded (preferably adhesive bonded or adhesive and heat bonded) to the insert with an adhesive or a further adhesive layer that is positioned or sandwiched between the protective strip(s) and the insert interconnecting protective strip(s) and the insert.

Preferably, the single piece frame is (preferably adhesive bonded or adhesive and heat bonded) to the insert with an adhesive or a further adhesive layer that is positioned or sandwiched between the protective strip(s) and the insert interconnecting protective strip(s) and the insert.

Preferably, only the edges or perimeter of the insert is unexposed or configured to be unexposed on its surface that is configured to face towards the user's body.

Preferably, inner more layer and the second adhesive layer only cover and the perimeter of the insert on its surface that is configured to face towards the user's body.

Preferably, the inner more layer comprises a centre portion extending longitudinally from a top end of the article to a bottom end of the article.

Preferably, the inner more layer comprises a first wing and a second wing each located between the top end of the article and the bottom end of the article, the first wing extending away from the centre portion of the inner more layer in a first direction and the second wing extending from the centre portion of the inner more layer in a second direction that is opposite the first direction.

Preferably, the first wing and the second wing of the inner more layer each comprise a fastening member for releasable coupling with the undergarment.

Preferably, the article comprises a fastening means for releasable coupling with the undergarment.

Preferably, the fastening means is a strap.

Preferably, the article is a urine incontinence absorbing and retaining article (e.g., urine incontinence pouch).

Preferably, the article is a menstrual fluid article (e.g., female hygiene article or menstrual hygiene article).

Preferably, the article is a menstrual fluid article (e.g., female hygiene article or menstrual hygiene article) that is configured to be positioned adjacent a person's genital for receiving menstrual fluid from genital area of the person.

In a second aspect, the present invention resides in a reusable moisture retaining article of or for use with an undergarment to be positioned adjacent a body of a user and to absorb and retain moisture from the user, the article comprising:
a reusable moisture absorbing and retaining insert at least a portion of which is exposed or configured to be exposed to define an inner layer of the article, the inner layer being configured to be located adjacent the user's body and facing towards the user's body for being in bodily contact with the user to directly absorb and preferably retain the moisture from the user;
an outer cover that is formed or assembled to define an outer layer of the article; and
an adhesive layer that is or at least a portion of which is positioned or sandwiched between a surface of the insert that is configured to face towards the outer cover and a surface of the outer cover that is configured to face towards the insert thereby interconnecting the insert and the outer cover, the insert and the outer cover are bonded together by the adhesive layer by applying a compressive force.

Preferably, the insert and the outer cover are bonded together by applying a compressive force to at least the insert and the outer cover and by applying heat and adhesive bonding to bond the insert and outer cover together using the first adhesive layer.

Preferably, the article further comprises:
a protective layer that is waterproof;
an adhesive layer that is a second adhesive layer; and
an inner more layer;
wherein the protective layer is positioned on a surface of the insert that is configured to face towards the user's body, at least a portion of the second adhesive layer is located between the protective layer and the inner more layer interconnecting at least the protective layer and the inner more layer, and the protective layer is bonded with the inner more layer and the insert (preferably by adhesive bonding or by adhesive and heat bonding).

Preferably, at least a further portion of the second adhesive layer is positioned between the insert and the inner more layer for interconnecting the insert and the inner more layer to bond the insert with inner more layer using the second adhesive layer.

Preferably, the protective layer is formed as at least one protective strip.

Preferably, the protective layer is a waterproof layer.

Preferably, the at least one protective strip is positioned or sandwiched between the insert and the second adhesive layer on the surface of insert that is configured to face towards the user's body.

Preferably, the protective layer is formed as at least two protective strips.

Preferably, the at least two protective strips are positioned or sandwiched between the insert and the second adhesive layer on the surface of insert that is configured to face towards the user's body and along at least two edges of the insert.

Preferably, the protective layer is formed as a single piece frame.

Preferably, the single piece frame is positioned or sandwiched between the insert and the second adhesive layer on the surface if the insert that is configured to face towards the user's body and long at least two edges (preferably, all edges) of the insert.

Preferably, the article is leakproof.

Preferably, the insert and the outer cover are bonded together by the adhesive layer by applying a compressive force and preferably compressive force and heat on the article.

Preferably, the compressive force is applied to the article.

Preferably, the compressive force is applied to the insert and the outer cover.

Preferably, the insert and the outer cover are bonded together by applying the adhesive and heat bonding.

Preferably, the heat bonding is applied at temperature between 120 to 200 degrees Celsius.

Preferably, the insert and the outer cover are bonded together only by the adhesive layer.

Preferably, the article is configured to be positioned adjacent a pudendal region of the user and to absorb and retain the moisture from the pudendal region of the user.

Preferably, the moisture is or comprises menstrual fluid and/or urine incontinence.

Preferably, the adhesive layer is coextensive with the insert.

Preferably, the adhesive layer is coextensive with the outer cover.

Preferably, the adhesive layer is coextensive with the insert and the outer cover.

Preferably, the adhesive layer is or comprises a bonding glue.

Preferably, the adhesive layer is non-woven.

Preferably, the adhesive layer is fused with waterproof textile.

Preferably, the outer cover is the outer most layer of the article.

Preferably, the outer cover is a decorative layer of the article.

Preferably, the outer cover is a protective layer to the insert and/or to the adhesive layer.

Preferably, the outer cover comprises or is made from woven and/or knitted textiles.

Preferably, the outer cover is coextensive with the insert.

Preferably, the outer cover is moisture resistant.

Preferably, a moisture resistant solution is applied to the outer cover.

Preferably, the outer cover is coated with a moisture resistant solution.

Preferably, the outer cover is a leakproof layer.

Preferably, the outer cover extends from the top of the article to the bottom of the article.

Preferably, the outer cover comprises one or more anti-slip prints at its outermost surface.

Preferably, one or more anti-slip prints are rubber prints.

Preferably, the anti-slip prints are made out of a silicon material.

Preferably, the silicon material glows in the dark.

Preferably, the insert is made from woven and/or knitted textile(s).

Preferably, the article further comprises an or the inner more layer located outwardly of the insert and bonded (preferably adhesive bonded or adhesive and heat bonded) to the insert using a second adhesive layer, the second adhesive layer being positioned or sandwiched between the insert and the inner more layer bonding (preferably adhesive bonding or adhesive and heat bonding) the inner more layer and the insert.

Preferably, the second adhesive layer is an adhesive strip.

Preferably, the second adhesive layer is or comprises a bonding glue.

Preferably, the insert is exposed to be an inner most layer of the article.

Preferably, the second adhesive layer is non-woven.

Preferably, the inner more layer is the inner most layer of the article.

Preferably, the inner more layer comprises or is made from woven or knitted textile(s).

Preferably, the inner more layer is moisture resistant.

Preferably, moisture resistant solution is applied to the inner more layer.

Preferably, the inner more layer is coated with moisture resistant solution.

Preferably, the inner more layer defines a window to allow the portion of insert to be exposed or configured to be exposed.

Preferably, the inner more layer comprises or is in the form of a first inner more strip and a second inner more strip and the second adhesive layer comprises or is in the form of a first adhesive strip and a second adhesive strip, wherein the first inner more strip is located outwardly of the insert and bonded (preferably adhesive bonded or adhesive and heat bonded) to the insert using the first adhesive strip and the second inner more strip is located outwardly of the insert and is bonded (preferably adhesive bonded or adhesive and heat bonded) to the insert using the second adhesive strip, the first adhesive strip being positioned or sandwiched between the insert and the first inner more strip bonding (preferably adhesive bonding or adhesive and heat bonding) the first inner more strip and the insert, and the second adhesive strip being positioned or sandwiched between the insert and the second inner more strip bonding (preferably adhesive bonding or adhesive and head bonding) the second inner more strip and the insert.

Preferably, each of the first and second adhesive strips is or comprises a bonding glue.

Preferably, each of the first and second adhesive strips is non-woven.

Preferably, each of the first and second inner more strips is the inner most layer of the article.

Preferably, each of the first and second inner more strips comprises or is made from woven or knitted textile(s).

Preferably, each of the first and second inner more strips is moisture resistant.

Preferably, moisture resistant solution is applied to each of the first and second inner more strips.

Preferably, each of the first and second inner more strips is coated with moisture resistant solution.

Preferably, a portion or a substantial portion of the insert is exposed or configured to be exposed to form the inner layer of the article.

Preferably, all of the insert is exposed or configured to be exposed to form the inner layer of the article save for its edges.

Preferably, the article comprises at least one protective strip that is waterproof and is positioned or sandwiched between the insert and the second adhesive layer on the surface of insert that is configured to face towards the user's body.

Preferably, the article comprises at least two protective strips that are waterproof and are positioned or sandwiched between the insert and the second adhesive layer on the surface of insert that is configured to face towards the user's body and along at least two edges of the insert.

Preferably, the protective strip(s) are positioned or sandwiched between the insert and the second adhesive layer on the surface of insert that is configured to face towards the user's body and along all edges/perimeter of the insert.

Preferably, the protective strips are formed as a single piece frame.

Preferably, the or each protective strip is bonded to the insert with an adhesive or a further adhesive layer that is positioned or sandwiched between the protective strip(s) and the insert interconnecting protective strip(s) and the insert.

Preferably, only the edges or perimeter of the insert is unexposed or configured to be unexposed on its surface that is configured to face towards the user's body.

Preferably, inner more layer and the second adhesive layer only cover the perimeter of the insert on its surface that is configured to face towards the user's body.

Preferably, the inner more layer comprises a centre portion extending longitudinally from a top end of the article to a bottom end of the article.

Preferably, the inner more layer comprises a first wing and a second wing each located between the top end of the article and the bottom end of the article, the first wing extending away from the centre portion of the inner more layer in a first direction and the second wing extending from the centre portion of the inner more layer in a second direction that is opposite the first direction.

Preferably, the first wing and the second wing of the inner more layer each comprise a fastening member for releasable coupling with the undergarment.

Preferably, the article comprises a fastening means for releasable coupling with the undergarment.

Preferably, the fastening means is a strap.

Preferably, the outer cover and the inner more layer are identical in shape and/or size.

Preferably, the article is a urine incontinence absorbing and retaining article (e.g., urine incontinence pouch).

Preferably, the article is a menstrual fluid absorbing and retaining article (e.g., female hygiene article or menstrual hygiene article).

Preferably, the article is a menstrual fluid absorbing and retaining article (e.g., female hygiene article or menstrual hygiene article) that is configured to be positioned adjacent a person's genital for receiving menstrual fluid from genital area of the person.

Preferably, the article is an incontinence article for female that is configured to be positioned adjacent a person's genital for receiving menstrual or other fluid from genital area of the person, including urine.

Preferably, the article is an incontinence pouch for men that is configured to be positioned adjacent a person's genital for receiving moisture from genital area of the person.

In a third aspect, the present invention resides in a method of constructing a reusable moisture retaining article of or for use with an undergarment to be positioned adjacent a body of a user and to absorb and retain a moisture body moisture from the user, the method comprising:
providing a reusable moisture absorbing and retaining insert at least a portion of which is exposed or configured to be exposed to define an inner layer of the article, the inner layer being configured to be located adjacent the user's body and facing towards the user's body for being in bodily contact with the user to directly absorb and retain the moisture from the user;
providing an outer cover that is configured to define an outer layer of the article;
positioning an adhesive layer or at least a portion of an/the adhesive layer that is a first adhesive layer between the insert and the outer cover, the adhesive layer being a first adhesive layer, wherein the first adhesive layer extends along at least a part of a surface area of the insert and the outer cover for interconnecting the insert and the outer cover;
applying a compressive force to at least the insert and the outer cover; and
applying heat and adhesive bonding thereby bonding the insert and the outer cover together using the first adhesive layer;
the method further comprising:
   providing an inner more layer;
   providing a protective layer that is waterproof;
   providing a further adhesive layer that is a second adhesive layer;
   positioning the protective layer on a surface of the insert that is configured to face towards the user's body;
   positioning the second adhesive layer so that at least a portion of the second adhesive layer is located between the protective layer and the inner more layer for interconnecting at least the protective layer and the inner more layer; and
   applying a further compressive force to at least the insert and the at least one inner more layer; and
   applying a heat and adhesive bonding thereby bonding the protective layer with the inner more layer and the insert.

Preferably, the method comprises positioning at least a further portion of the second adhesive layer between the insert and the inner more layer for interconnecting the insert and the inner more layer thereby bonding the insert with inner more layer using the second adhesive layer.

Preferably, the heat bonding is applied at temperature between 120 to 200 degrees Celsius.

Preferably, the method comprises positioning each layer of the article so that they are substantially parallel to each other.

Preferably, the adhesive layer is coextensive with the insert and/or the outer cover.

Preferably, the first and/or second adhesive layer is or comprises a bonding glue.

Preferably, the first and/or second adhesive layer is non-woven.

Preferably, the first and/or second adhesive layer is fused with waterproof textile.

Preferably, the outer cover is the outer most layer of the article.

Preferably, the outer cover is a decorative layer of the article.

Preferably, the outer cover is a protective layer to the insert.

Preferably, at least one of the outer cover and the inner more layer comprises or is made from woven and/or knitted textiles.

Preferably, at least one of the outer cover and the inner more layer is coextensive with the insert.

Preferably, at least one of the outer cover and the inner more layer is moisture resistant, a moisture resistant solution is applied to the outer cover and/or the outer cover is coated with a moisture resistant solution.

Preferably, the insert is made from woven and/or knitted textile(s).

Preferably, the second adhesive layer is formed as at least one adhesive strip.

Preferably, the inner more layer is the inner most layer of the article.

Preferably, the inner more layer comprises or is in the form of a first inner more strip and a second inner more strip and the second adhesive layer comprises or is in the form of a first adhesive strip and a second adhesive strip, wherein the method comprises: locating the first inner more strip outwardly of the insert and bonding the first inner more strip to the insert and the protective layer using the first adhesive strip; and locating the second inner more strip outwardly of the insert and bonding the second inner more strip to the insert and the protective layer using the second adhesive strip, wherein the method further comprises positioning or sandwiching at least a portion of the first adhesive strip between the insert and the first inner more strip thereby bonding the first inner more strip and the insert, and positioning or sandwiching at least a portion of the second adhesive strip between the insert and the second inner more strip thereby bonding the second inner more strip and the insert.

Preferably, the inner more layer defines a window to allow the portion of insert to be exposed or configured to be exposed.

Preferably, the method comprises exposing all of the insert from the window to form the inner layer of the article save for its edges.

Preferably, the protective layer is formed as at least one protective strip and the method comprises positioning or sandwiching the at least one protective strip between the insert and the second adhesive layer on the surface of insert that is configured to face towards the user's body.

Preferably, the protective layer is formed as at least two protective strips and the method comprises positioning or sandwiching the at least two protective strips between the insert and the second adhesive layer on the surface of insert that is configured to face towards the user's body and along at least two edges of the insert.

Preferably, the method comprises positioning or sandwiching the protective strip(s) between the insert and the second adhesive layer on the surface of the insert that is configured to face towards the user's body and along all edges/perimeter of the insert.

Preferably, the protective layer is formed as a single piece frame.

Preferably, the protective layer is a waterproof layer.

Preferably, the article is a urine incontinence absorbing and retaining article, or a menstrual fluid absorbing or retaining article.

One or more statements relating to the first or second aspect may equally apply to the third aspect.

In a fourth aspect, the present invention resides in a reusable moisture retaining article of or for use with an undergarment to be positioned adjacent a body of a user and to absorb and retain moisture from the body of the user, the article being a reusable article and comprising:
a reusable moisture absorbing and retaining insert at least a portion of which is exposed or configured to be exposed to define an inner layer of the article, the inner layer being configured to be located adjacent the user's body and facing towards the user's body for being in bodily contact with the user to directly absorb and retain the moisture from the body of the user;
an outer cover that is formed or assembled to define an outer layer of the article; and
an adhesive layer that is a first adhesive layer, the adhesive layer or at least a portion of the adhesive layer being is positioned or sandwiched between a surface of the insert that is configured to face towards the outer cover and a surface of the outer cover that is configured to face towards the insert thereby interconnecting the insert and the outer cover, the insert and the outer cover are bonded together by applying a compressive force to at least the insert and the outer cover and by applying heat and adhesive bonding to bond the insert and outer cover together using the first adhesive layer;
the article further comprising:
   a protective material that is waterproof;
   an adhesive layer that is a second adhesive layer; and
   an inner more layer;
   wherein the protective layer is positioned on a surface of the insert that is configured to face towards the user's body, at least a portion of the second adhesive layer is located between the protective layer and the inner more layer interconnecting at least the protective layer and the inner more layer, and the protective layer bonded with the inner more layer and the insert.

Preferably, the heat bonding is applied at temperature between 120 to 200 degrees Celsius.

Preferably, a further portion of the second layer is located between the insert and the inner more layer for interconnecting and bonding the insert and the inner more layer.

Preferably, the protective layer is a waterproof layer.

Preferably, the article is configured to be positioned adjacent a pudendal region of the user and to absorb and retain the moisture from the pudendal region of the user.

Preferably, the first and/or second adhesive layer is coextensive with the insert and/or the outer cover.

Preferably, the first and/or second adhesive layer is or comprises a bonding glue.

Preferably, the first and/or second adhesive layer is non-woven.

Preferably, the first and/or second adhesive layer is fused with waterproof textile.

Preferably, the outer cover is the outer most layer of the article.

Preferably, the outer cover is a decorative layer of the article.

Preferably, at least one of the outer cover and the inner more layer comprises or is made from woven and/or knitted textiles.

Preferably, at least one of the outer cover and the inner more layer is coextensive with the insert.

Preferably, at least one of the outer cover and the inner more layer is moisture resistant, a moisture resistant solution is applied to the outer cover and/or the outer cover is coated with a moisture resistant solution.

Preferably, the outer cover is a leakproof layer.

Preferably, the outer cover extends from the top end of the article to the bottom end of the article.

Preferably, the outer cover comprises one or more anti-slip prints at its outermost surface.

Preferably, one or more anti-slip prints are rubber prints.

Preferably, the one or more anti-slip prints are made out of a silicon material.

Preferably, the silicon material glows in the dark.

Preferably, the insert is made from woven and/or knitted textile(s).

Preferably, the second adhesive layer is formed as at least one an adhesive strip.

Preferably, the inner more layer comprises or is in the form of a first inner more strip and a second inner more strip and the second adhesive layer comprises or is in the form of a first adhesive strip and a second adhesive strip, wherein the first inner more strip is located outwardly of the insert and bonded to the insert using the first adhesive strip and the second inner more strip is located outwardly of the insert and is bonded to the insert using the second adhesive strip, at least a portion of the first adhesive strip being positioned or sandwiched between the insert and the first inner more strip bonding the first inner more strip and the insert, and at least a portion of the second adhesive strip being positioned or sandwiched between the insert and the second inner more strip bonding the second inner more strip and the insert.

Preferably, the inner more layer defines a window to allow the portion of insert to be exposed or configured to be exposed.

Preferably, all of the insert is exposed or configured to be exposed to form the inner layer of the article save for its edges.

Preferably, the protective layer is formed as at least one protective strip that is positioned or sandwiched between the insert and the second adhesive layer on the surface of insert that is configured to face towards the user's body.

Preferably, the protective layer is formed as at least two protective strips that are waterproof and are positioned or sandwiched between the insert and the second adhesive layer on the surface of insert that is configured to face towards the user's body and along at least two edges of the insert.

Preferably, the protective strip(s) are positioned or sandwiched between the insert and the second adhesive layer on the surface of insert that is configured to face towards the user's body and along all edges/perimeter of the insert.

Preferably, the at least protective layer is formed as a single piece frame.

Preferably, the protective layer is bonded to the insert with an adhesive or a further adhesive layer that is a third adhesive layer that is positioned or sandwiched between the protective layer and the insert interconnecting protective layer and the insert.

Preferably, only edges or perimeter of the insert is unexposed or configured to be unexposed on its surface that is configured to face towards the user's body.

Preferably, inner more layer and the second adhesive layer only cover and the perimeter of the insert on its surface that is configured to face towards the user's body.

Preferably, the inner more layer comprises a centre portion extending longitudinally from a top end of the article to a bottom end of the article.

Preferably, the inner more layer comprises a first wing and a second wing each located between the top end of the article and the bottom end of the article, the first wing extending away from the centre portion of the inner more layer in a first direction and the second wing extending from the centre portion of the inner more layer in a second direction that is opposite the first direction.

Preferably, the first wing and the second wing of the inner more layer each comprise a fastening member for releasable coupling with the undergarment.

Preferably, the article comprises a fastening means for releasable coupling with the undergarment.

Preferably, the fastening means is a strap.

Preferably, the protective layer is a waterproof layer.

One or more statements relating to the first, second or third aspect may equally apply to the fourth aspect.

In a fifth aspect, the present invention resides in a reusable menstrual fluid absorbing and retaining article, the article formed as an assembly of a plurality of layers, wherein each of the layers are bonded together by an adhesive when a compressive force is applied on the article.

Preferably, the layers of the assembly are bonded together only by the adhesive when a compressive force is applied on the article.

Preferably, the article comprises:
a reusable a moisture absorbing and retaining insert at least a portion of which is exposed or configured to be exposed to define an inner layer of the article, the inner layer being located adjacent a user's body and facing towards the user's body for being in bodily contact with the user to directly absorb and retain the menstrual fluid from the user;
an outer cover that is formed or assembled to define an outer layer of the article; and
an adhesive layer that is positioned or sandwiched between a surface of the insert that is configured to face towards the outer cover and a surface of the outer cover that is configured to face towards the insert thereby interconnecting the insert and the outer cover, the insert and the outer cover are bonded together by the adhesive layer by applying a compressive force on the article.

One or more statements relating to the first, second, third or fourth aspect may equally apply to the fifth aspect.

In a sixth aspect, the present invention resides in a reusable urine incontinence absorbing and retaining article, the article formed as an assembly of a plurality of layers, wherein each of the layers are bonded together by an adhesive when a compressive force is applied on the article.

Preferably, the layers of the assembly are bonded together only by the adhesive when a compressive force is applied on the article.

Preferably, the article comprises:
a reusable insert at least a portion of which is exposed or configured to be exposed to define an inner layer of the article, the inner layer being located adjacent a user's body and facing towards the user's body for being in bodily contact with the user to directly absorb and retain the menstrual fluid from the user;
an outer cover that is formed or assembled to define an outer layer of the article; and
an adhesive layer, wherein the adhesive layer or at least the portion of the adhesive layer is positioned or sandwiched between a surface of the insert that is configured to face towards the outer cover and a surface of the outer cover that is configured to face towards the insert thereby interconnecting the insert and the outer cover, the insert and the outer cover are bonded together by the adhesive layer by applying a compressive force on the article.

One or more statements relating to the first, second, third or fourth aspect may equally apply to the sixth aspect.

In a seventh aspect the present invention may broadly be said to be a garment that includes the article as herein described.

Preferably, the garment is an underwear.

Preferably, the garment is a male underwear.

Preferably, the garment is a female underwear.

Preferably, the garment is able to receive the article in a dedicated location of the garment.

One or more statements relating to the first, second, third or fourth aspect may equally apply to the seventh aspect.

In an eighth aspect, the present invention resides in a method of constructing a reusable menstrual fluid absorbing and retaining article, the article formed as an assembly of a plurality of layers, wherein the method comprises applying adhesive or adhesive layers on each of the layers to be bonded and bonding the layers together by applying a compressive force on the article.

One or more statements relating to the first, second, third or fourth aspect may equally apply to the eighth aspect.

In a ninth aspect, the present invention may be said to be a method of constructing a reusable urine incontinence absorbing and retaining article, the article formed as an assembly of a plurality of layers, wherein the method comprises applying adhesive or adhesive layers on each of the layers to be bonded and bonding the layers together by applying a compressive force on the article.

One or more statements relating to the first, second, third or fourth aspect may equally apply to the ninth aspect.

In a tenth aspect, the present invention resides in an undergarment that comprises or incorporates an article that constructed using any of the method described above.

Preferably, the undergarment is an underwear.

Preferably, the undergarment is a male underwear.

Preferably, the undergarment is a female underwear.

In any of the above aspects, one or more statements relating to the any one aspect may equally apply to any other aspect.

As used hereinbefore and hereinafter, the term “and/or” means “and” or “or”, or both.

As used hereinbefore and hereinafter, “(s)” following a noun means the plural and/or singular forms of the noun.

As used hereinbefore and hereinafter and unless stated otherwise, the word ‘liquid’ in the context of this specification may include ‘liquid’ but may equally include ‘moisture’.

As used hereinbefore and hereinafter and unless stated otherwise, the word ‘moisture resistant’ in the context of this specification may also include ‘water resistant’.

As used hereinbefore and hereinafter and unless stated otherwise, the word ‘waterproof’ in the context of this specification may include ‘waterproof’ but may also include something that is impervious body moisture.

As used hereinbefore and hereinafter and unless stated otherwise, the word “moisture” in the context of this specification may include any fluid, liquid and/or moisture discharged from a human body and may also include menstrual fluid/discharges and/or urine.

As used hereinbefore and hereinafter and unless stated otherwise, ‘compressive force’ in the context of this specification may include compressive force but may also include compressive force applied together with heat at a suitable temperature for bonding purposes. The same rationale applies to ‘compression force’.

As used hereinbefore and hereinafter and unless stated otherwise, the word ‘retain’ in the context of this specification may mean ‘continue to hold or contain something’ but may also be capable of being released such as by evaporation, squeezing and wringing etc. The word ‘retain’ which is different from ‘absorb’ which in the context of the specification may mean ‘to take and/or draw’ in a liquid from the surface or space around’. Unless otherwise state, this rationale will also apply for the word(s) ‘retaining’ and/or ‘retention’ as used hereinbefore and hereinafter.

When used in claim and unless stated otherwise, the word ‘for’ is to be interpreted to mean only ‘suitable for’, and not for example, specifically ‘adapted’ or ‘configured’ for the purpose that is stated.

As used hereinbefore and hereinafter and unless stated otherwise, the word “FIG” or “FIG.” in the context of this specification means Figure.

As used hereinbefore and hereinafter and unless stated otherwise, the word “FIGs” or “FIGs.” in the context of this specification means Figures.

It is known, to those with skill in the art of patenting, that the word ‘substantially’ can, in some instances, be used to broaden a term. It should be stated that, in the present application, use of the word ‘substantially’ with a term, to define a (characterizing) feature(s), gets all the benefit (i.e the benefit of any broadening) afforded by use of the word ‘substantially’, and also includes within its scope the feature(s) being that term exactly (without broadening). For example, if a feature is described/defined in the present application as being ‘substantially vertical’, then that includes, within its scope, the features being ‘close’ to vertical (in so far as the word ‘substantially’ is deemed to broaden the term ‘vertical’), and also includes within its scope the features being ‘exactly’ vertical.

### BRIEF DESCRIPTION OF THE DRAWINGS

The invention will now be described by way of example only and with reference to the drawings in which:

FIG. 1 is an exploded view of an article according to one embodiment of the present invention.

FIG. 2A is an outer view of the article of FIG. 1 in an assembled form

FIG. 2B is an inner view of the article of FIG. 1 in an assembled form

FIG. 2C is an outer view of an article according to one alternative embodiment of the present invention.

FIG. 2D is an inner view of an article according to one alternative embodiment of the present invention.

FIG. 3 is the embodiment of FIG. 1 with waterproof protective strips.

FIG. 4 is the embodiment of FIG. 1 with another form of waterproof protective strips.

FIG. 5A is the inner view of embodiment of FIG. 3 in an assembled form.

FIG. 5B is the inner view of embodiment of FIG. 4 in an assembled form.

FIG. 6A is an exploded view of an article according to another embodiment of the present invention.

FIG. 6B is the embodiment of FIG. 6A with waterproof protective strips.

FIG. 6C is the embodiment of FIG. 6A with another form of waterproof protective strips.

FIG. 7A is the inner view of embodiment of FIG. 6A in an assembled form.

FIG. 7B is the inner view of embodiment of FIG. 6B in an assembled form.

FIG. 7C is the inner view of embodiment of FIG. 6C in an assembled form.

FIG. 8 shows an article according to another embodiment of the present invention.

FIGS. 8A-D are cut through views along A-A axis of FIG. 8 showing several different possible configurations of the different layers.

FIG. 9 shows B-B axis of the article of FIG. 8.

FIGS. 9A-D are cut through views along B-B axis of FIG. 9 showing several different possible configurations of the different layers.

FIG. 10A is an exploded view of an article according to a further embodiment of the present invention.

FIG. 10B is the embodiment of FIG. 10A with waterproof protective strips.

FIG. 11A is an outer view of the article of FIG. 10A in an assembled form.

FIG. 11B is an inner view of the article of FIG. 10A in an assembled form.

FIG. 11C is an outer view of the article of FIG. 10B in an assembled form.

FIG. 11D is an inner view of the article of FIG. 10B in an assembled form.

FIG. 12A shows a front view of the men's underwear comprising the article according to a further embodiment of the present invention that is same or similar to the article of FIG. 10A or FIG. 10B.

FIG. 12B shows a rear view of the underwear of FIG. 10A or FIG. 10B.

FIG. 12C shows an exploded view of the underwear of FIG. 12A.

FIG. 12D shows the embodiment of FIG. 12C but with waterproof protective strips.

FIG. 13 shows an article according to a yet another embodiment of the present invention.

FIGS. 13A-D are cut through views along C-C axis of FIG. 13 showing several different possible configurations of the different layers.

FIG. 14 shows D-D axis of the article of FIG. 13.

FIGS. 14A-D are cut through views along D-D axis of FIG. 14 showing several different possible configurations of the different layers.

### DETAILED DESCRIPTION

In accordance with an exemplary embodiment of the present invention, a reusable moisture retaining article is provided. This article is reusable and can be washed and dried additionally optionally also be printable, non-fraying and/or dimensionally stable. The fabric material may be highly absorbent and has a quick wicking and has a moisture retention ability. The moisture may be an exudate from a human body such a but not limited to menstrual fluid and/or urine incontinence.

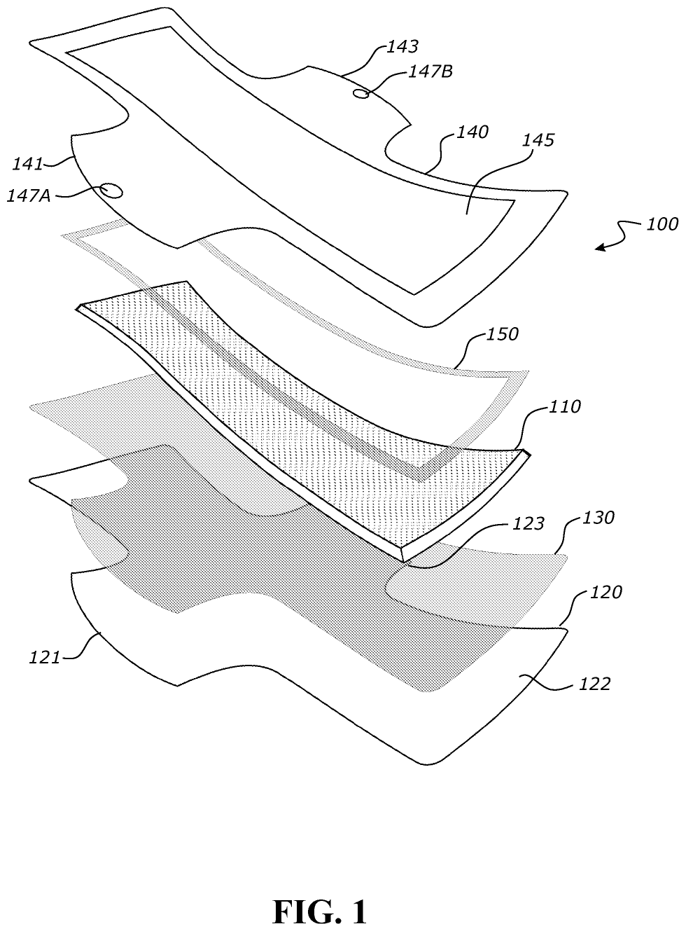

Reference will now be made to the accompanying drawings in which FIG. 1 shows an exploded view of the reusable moisture retaining article 100 according to one embodiment of the present invention.

The article 100 is of or for use with an undergarment to be positioned adjacent a body of a user and to receive moisture from the body of the user. The user may be a person wearing the article. The article 100 may be used as a menstrual fluid absorbing and retaining article (also referred to as a “female hygiene article” or “menstrual hygiene article”, “female liner” or “menstrual pad”). The article 100 may also be suitable for absorbing and retaining any other moisture from the body of the user.

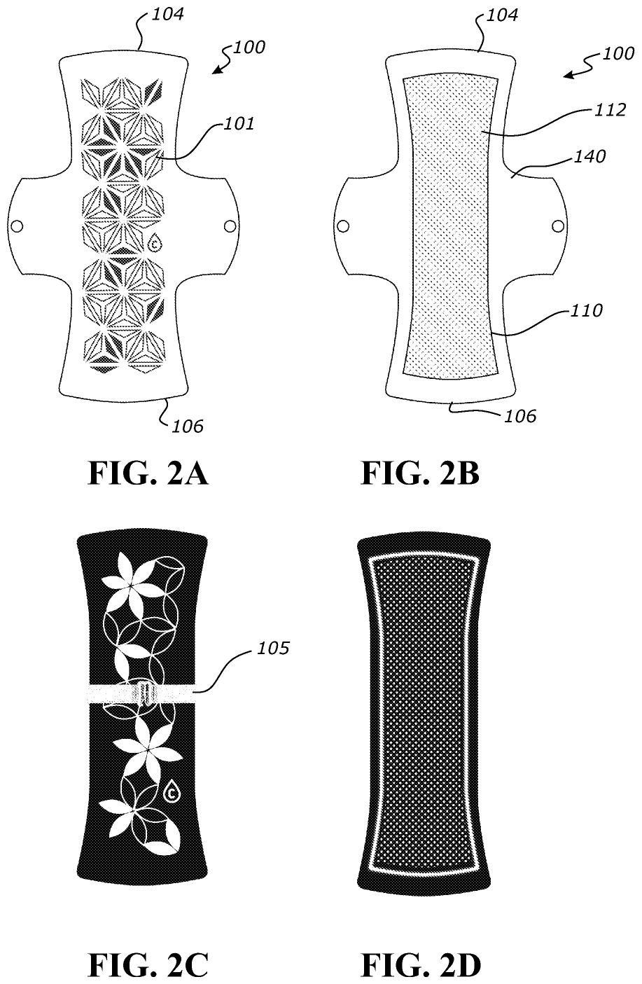

The article 100 comprises a reusable moisture absorbing and retaining insert 110 (hereinafter “insert”). As shown in FIG. 2B at least a portion 112 of the insert 110 is exposed to define an inner layer of the article 100. The inner layer of the article is configured to be located adjacent the user's body and facing towards the user's body for being in bodily contact with the user to directly receive the moisture from the body of the user.

Portion 112 may optionally be covered by a removable material e.g., removable film or similar that when such removable material is removed, portion 112 is exposed. This helps prevent the contamination of portion 112, which is the inner layer of the article that is configured to be in bodily contact with the user.

The article 100 comprises multiple layers as shown in FIG. 1. These multi-layers are at least a reusable insert 110, an outer cover 120 and an adhesive layer 130.

As mentioned above, at least a portion 112 of the reusable insert 110 is exposed or configured to be exposed to define an inner layer of the article 100. The inner layer (i.e., the portion that is exposed or configured to be exposed) is configured to be located adjacent the user's body to be in bodily contact with the user to directly absorb and retain the moisture from the body of the user. If the article 100 is used as a menstrual fluid absorbing and retaining article, the moisture of the body of the user that is absorbed and retained through the exposed portion 112 of the insert 110 will be menstrual fluid/menstrual discharge.

The outer cover 120 is formed or assembled to define an outer layer of the article.

The adhesive layer 130 is positioned or sandwiched between the insert 110 and the outer cover 120 thereby interconnecting the insert and the outer cover. More specifically, the adhesive layer 130 is positioned or sandwiched between a surface of the insert 110 that is configured to face towards the outer cover 120 and a surface of the outer cover 120 that is configured to face towards the insert 110 thereby interconnecting the insert 110 and the outer cover 120.

The insert 110 and the outer cover 120 are bonded together using the adhesive layer 130.

A skilled person will appreciate that with such a configuration the insert 110 and the outer cover 120 can be bonded together (e.g., adhesive and/or heat bonding) by simply applying a compression force (i.e., compressive force). The compressive force may be applied on the article 100, more specifically on the insert 110 and the outer cover 120 of the article 100. Preferably, the heat bonding is applied at temperature between 120 to 200 degrees Celsius. Such temperature range correlates to the melting point of the textile layers (e.g. insert and the outer layer) thereby allowing the layers to bond together.

In one embodiment, with such a configuration the insert 110 and the outer cover 120 is bonded by adhesive and heat bonding together by applying compression. Such configuration and bonding technique eliminate or at least reduce stitching and/or other complex process for bonding the layers together thereby significantly reducing the manufacturing process and costs. Also, complex machinery will not be required for manufacturing the article 100.

The article 100 may be configured to be positioned adjacent a pudendal region of the user and to receive, absorb and retain the moisture from the pudendal region of the user. The article 100 may be or may also be used for receiving or absorbing the urine incontinence. Such received and absorbed moisture may be retained by the article 100.

The adhesive layer 130 may be coextensive with the insert 110 and/or the outer cover 120. The adhesive layer 130 may be or may comprise a bonding glue. The adhesive layer 130 may be non-woven. Preferably, the adhesive layer 130 may be fused with waterproof textile. This further enhances the leakproof properties of the article 100 thereby providing ultimate protection to the user.

The outer cover 120 may be the outer most layer of the article 100. As shown in FIG. 2A, the outer cover 120 may be a decorative layer of the article 100. It may be function as a layer to protect the insert 110 and/or to the adhesive layer 120. The outer cover 120 may be moisture resistant. Preferably, a moisture resistant solution is applied to the outer cover 120. The outer cover 120 may be coated with a moisture resistant solution.

As shown in FIG. 2A, the outer cover 120 may comprise anti-slip prints 101 (e.g., anti-slip rubber prints) at outermost surface to avoid movements when the article 100 is placed inside an undergarment such as an underwear. In certain embodiments, the anti-slip prints 101 may be made out of a silicon material, preferably silicon material that glows in the dark.

The insert 110 may comprise or may be made from a woven and/or knitted textile(s). Preferably the insert 110 may be a moisture retention textile assembly as described in PCT Publication number: WO2020121176A1, the entirety of which is herein incorporated by reference.

As shown in FIG. 1, there may also be provided an inner more layer 140 (also may be referred to as innermore layer) that is located outwardly of the insert 110. A second adhesive layer 150 may be positioned or sandwiched between the insert 110 and the inner more layer 140. Therefore, the second adhesive layer 150 may interconnect the insert 110 and the inner more layer 140. Application of compressive force may allow the insert 110 and the inner more layer 140 to be bonded together. Preferably, the heat bonding may be applied in addition to the adhesive bonding to bond insert 110 and the inner more layer 140. The heat bonding may be applied at temperature between 120 to 200 degrees Celsius, which temperature range is advantageous at least for same or similar reason as already explained above.

The second adhesive layer 150 may be an adhesive strip. The second adhesive layer 150 is or may comprise a bonding glue. The second adhesive layer 150 may be non-woven. The composition of the second adhesive layer 150 may be same as the adhesive layer 130.

The inner more layer 140 may be the inner most layer of the article 100. The inner more layer 140 may be moisture resistant. Preferably, a moisture resistant solution may be applied to the inner more layer 140. Preferably, the inner more layer 140 may be coated with moisture resistant solution. Preferably, the insert 110 is exposed to be an inner most layer of the article 100.

The inner more layer 140 may define a window 145 through which the portion 112 of insert 110 is exposed or configured to be exposed. As shown, a substantial portion 112 of the insert 110 is exposed or configured to be exposed to form the inner layer of the article 100. In this example shown, all of the insert 110 is exposed or configured to be exposed to form the inner layer of the article 100 except its edges. Hence, on its surface that is configured to face towards the user's body, only the edges or perimeter of the insert 110 may be unexposed or configured to be unexposed. The inner more layer 140 and the second adhesive layer 150 may only cover the perimeter of the insert 110 on its surface that is configured to face towards the user's body.

The inner more layer 140 may comprise a centre portion extending longitudinally from a top end 104 of the article to a bottom end 106 of the article 100. The inner more layer 140 may comprise a first wing 141 and a second wing 143 each located between the top end 104 and the bottom end 106 of the article 100. The first wing 141 may extend away from the centre portion of the inner more layer 140 in a first direction and the second wing 143 may extend from the centre portion 142 of the inner more layer 140 in a second direction that is opposite the first direction. The window 145 may be located in the centre portion 142.

The first wing 141 and the second wing 143 of the inner more layer 140 may each comprise a fastening member 147A, 147B (e.g., buttons) for releasable coupling with the undergarment such as an underwear.

The outer cover 120 and the inner more layer 140 may be identical in shape and/or size, except that centre portion of the inner more layer 140 comprises a window 145 whereas no such window appears in the outer cover 120. Therefore, the outer cover 120 may comprise a centre portion 122 extending longitudinally from a top end 104 of the article to a bottom end 106 of the article 100. The outer cover 120 may comprise a first wing 121 and a second wing 123 each located between the top end 104 the bottom end 106 of the article 100. The first wing 121 may extend away from the centre portion 122 of the outer cover 120 in a first direction and the second wing 123 may extend from the centre portion 122 of the outer cover 120 in a second direction that is opposite the first direction.

The insert 110 may extend longitudinally from a top end 104 of the article to a bottom end 106 of the article 100. The insert 110 may be of the same size as the centre portion 122 of the outer cover 120 and/or the centre portion of the inner more layer 150.

The adhesive layer 130 may be of the same shape or size as the outer cover 120.

The article 100 may be of elongate shape with each of the layers extending longitudinally the top end 104 and the bottom end 106 and without any wing. Such article may optionally comprise a strap 105 or similar fastening means for releasable coupling with the undergarment. FIG. 2C shows an example of such an embodiment.

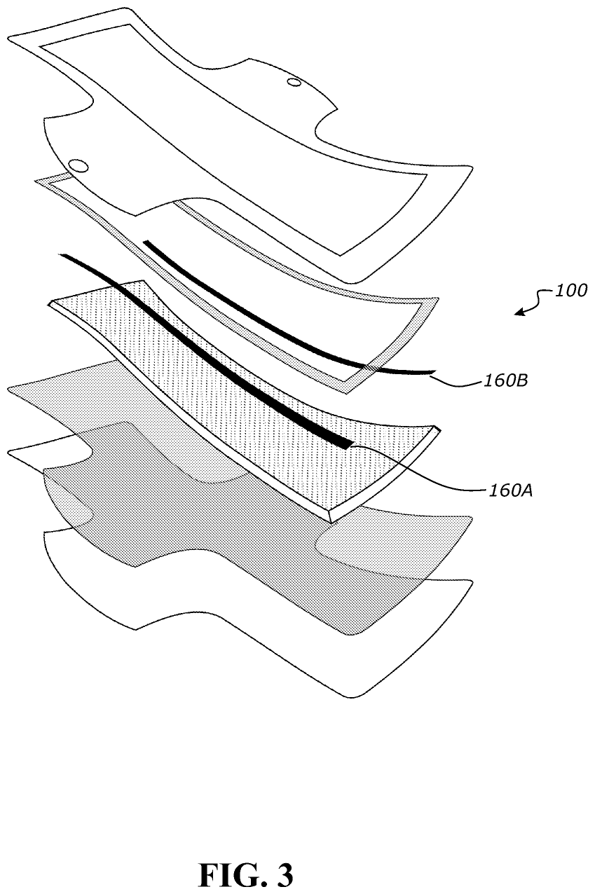

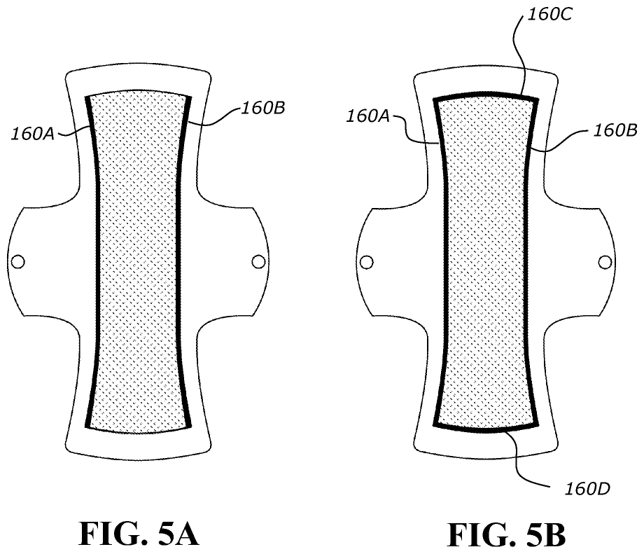

The article 100 may comprise a protective layer (which can also be referred to as a waterproof layer). The protective layer can be a strip layer. The protective layer may be in the form of at least one protective strip that is waterproof. As shown in FIGS. 3 and 5A, the protective layer may be in the form of two protective strips 160A, 160B that are waterproof. The protective strips may be made from woven polyester or polyamide. The protective strips 160A, 160B may be positioned or sandwiched between the insert and the second adhesive layer. The protective strips 160A, 160B may be positioned on the surface of insert 120 that is configured to face towards the user's body along at least two longitudinal edges/sides of the insert 110. Such protective strips 160A, 160B can avoid any moisture transfer from insert 120 beyond those two edges/sides of the insert 110, thereby providing technical advantage.

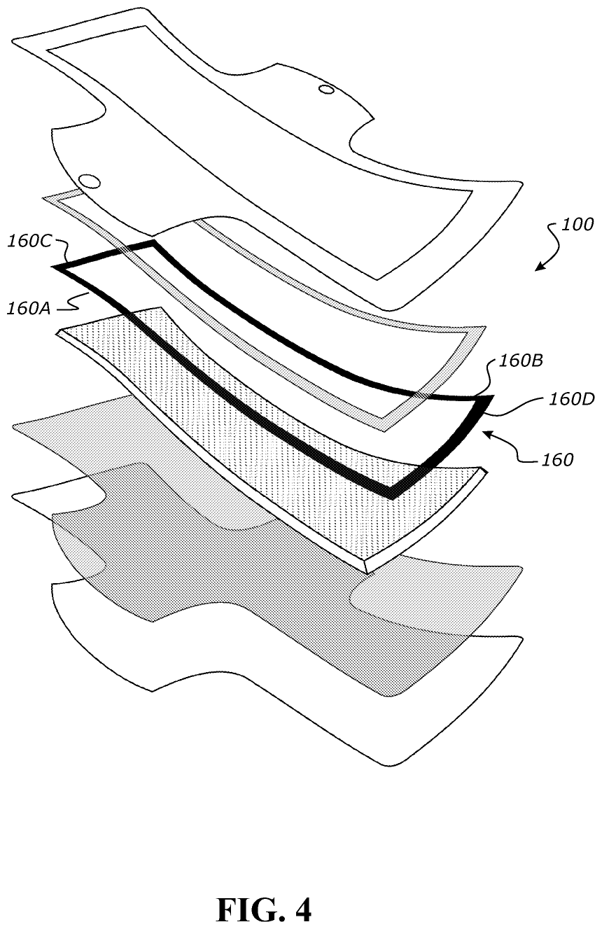

Alternatively, as shown in FIGS. 4 and 5B, the protective layer may be in the form of protective strips 160A, 160B, 160C, 160D that may be positioned on the surface of insert 150 that is configured to face towards the user's body along all of the edges/perimeter of the insert. Such protective strips 160A, 160B, 160C, 160D can avoid any moisture transfer from insert 110 beyond any of the edges/sides. Protective strips 160A, 160B, 160C, 160D may be separate pieces or may be integrally formed to form a single piece frame 160, therefore may be a single piece protective strip or a single piece protective layer.

Each layer of the article 100 may be positioned so that they are substantially parallel to each other.

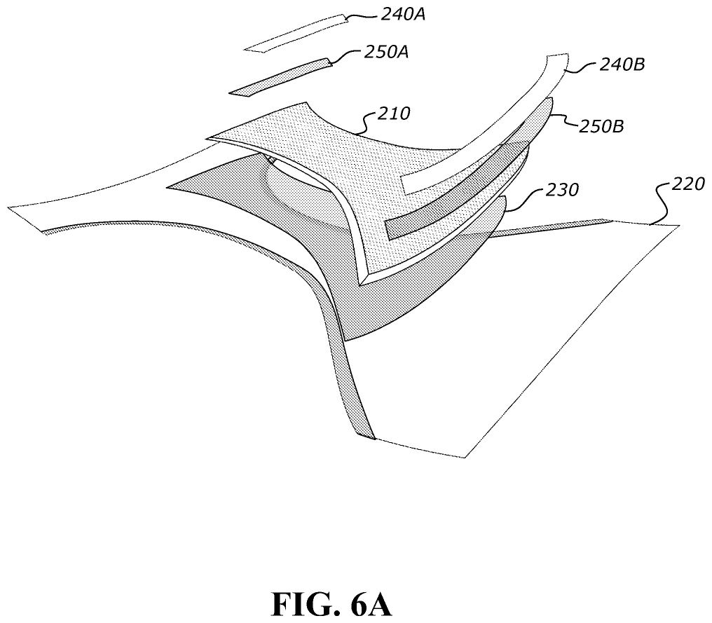

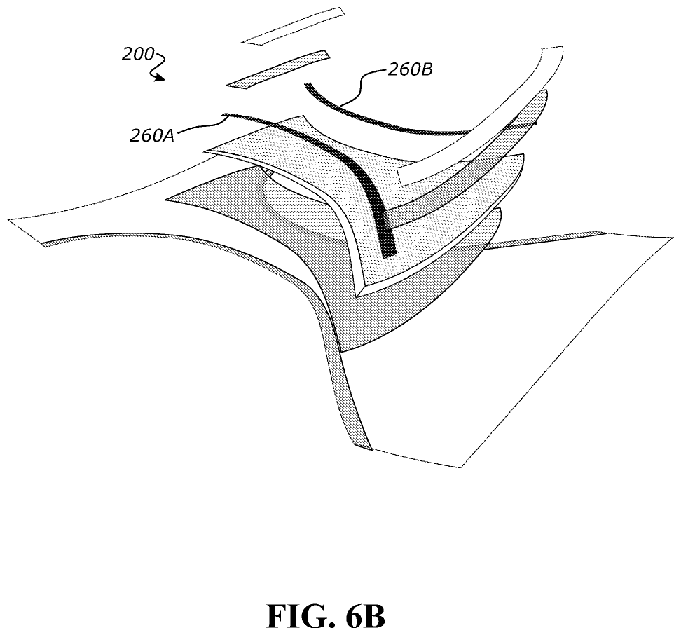

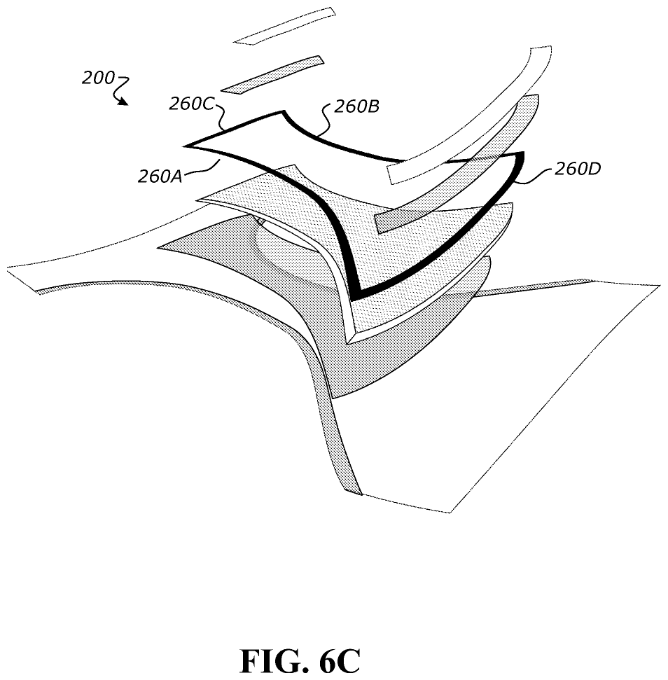

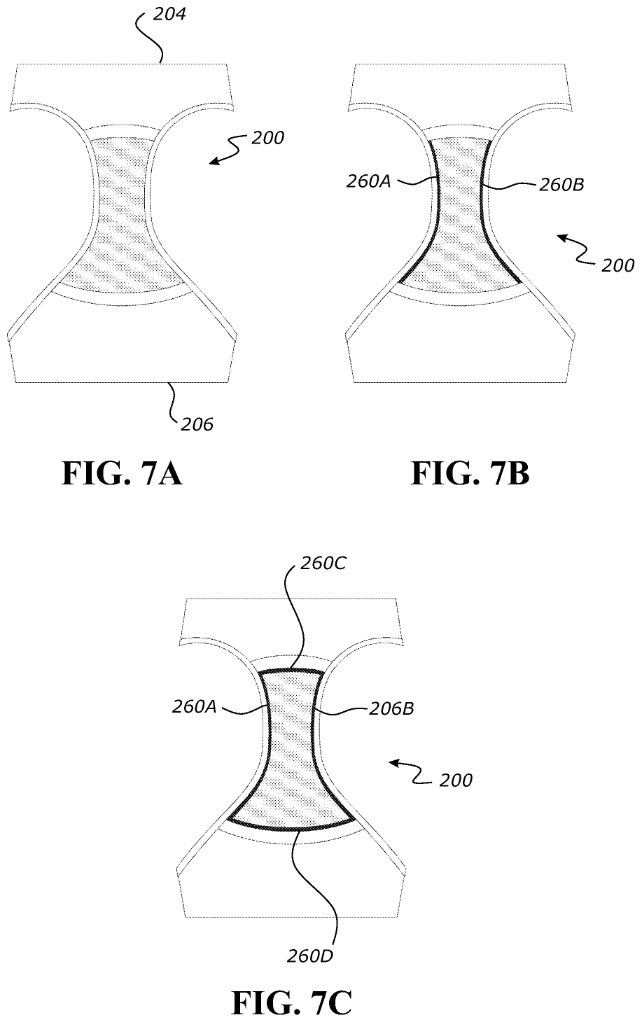

FIGS. 6A-6C and 7A-7C show an article 200 according to another preferred embodiment of the present invention. The article 200 in this example is a female underwear or a part of the female underwear.

The article 200 of this example is similar in most aspects to the article 100 described above and the differences can be identified by comparing FIG. 1 with FIG. 6A, FIG. 2B with FIG. 7A, FIG. 3 with FIG. 6B, FIG. 4 with FIG. 6C, FIG. 5A with FIG. 7B and FIG. 5B with FIG. 7C. In FIGS. 6A-6C and 7A-7C, the features that are similar to those shown in figures relating to article 100 are identified with the same reference numeral, incremented by 100. Most of the description of the article 100 of a preferred embodiment above, equally applies to the article 200 and therefore need not be described again in too much detail. Only the main differences will be discussed.

Here the outer cover 220 is a female underwear panel and may be made from knitted textiles and are preferably highly stretchable for comfort.

The adhesive layer 230 is positioned or sandwiched between the insert 210 and the outer cover 220 thereby interconnecting the insert and the outer cover.

The insert 210 and the outer cover 220 are bonded together using the adhesive layer 230. The bonding may be by application of heat bonding in addition to adhesive bonding. Preferably, the heat bonding is applied at temperature between 120 to 200 degrees Celsius.

The inner more layer is in the form of strips 240A, 240B that are located outwardly of the insert 210. A second adhesive layer are also in the form of adhesive strips 250A, 250B. Adhesive strip 250A may be positioned or sandwiched between the insert 210 and the inner more layer strip 240A. Similarly, adhesive strip 250B may be positioned or sandwiched between the insert 210 and the inner more layer strip 240B. Therefore, the adhesive strips 250A, 250B may interconnect the insert 210 and the inner more strips 240A, 240B.

The inner more strips 240A, 240B may be made from the same textiles as the outer cover 220.

The inner more strips 240A, 240B and the adhesive strips 250A, 250B may laterally extend along a first edge of the insert 210 that is proximal to the top end 204 of the article and a second edge that is proximal to the bottom end 206 of the article 200. More specifically, the inner more strip 240A and the adhesive strip 250A may laterally extend along a first edge of the insert 210 that is proximal to the top end 204 of the article 200. Similarly, the inner more strip 240B and the adhesive strip 250B may laterally extend along a second edge of the insert 210 that is proximal to the bottom end 206 of the article 200.

In some embodiments, the inner more strips 240A, 240B and adhesive strips 250A, 250B may be optional and therefore, the insert 210 may be the innermost layer of the article 200.

Other than the above mentioned differences and any other differences that are obvious from the figures, all other features of the article 200 including the properties of its various layers may be same as that described above with reference to article 100.

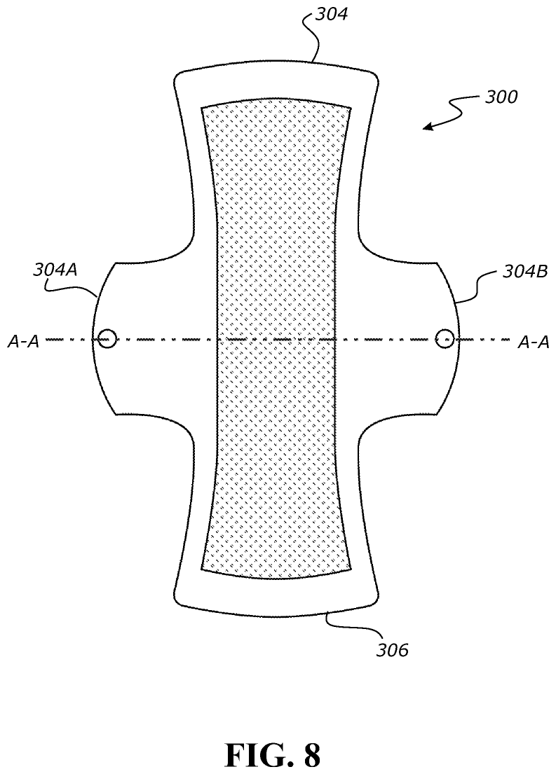

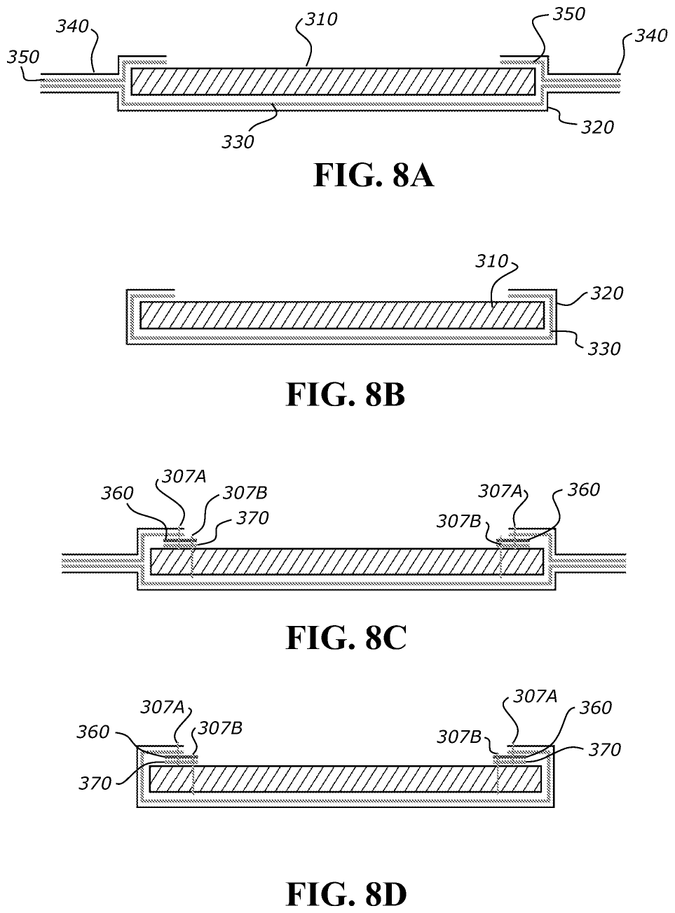

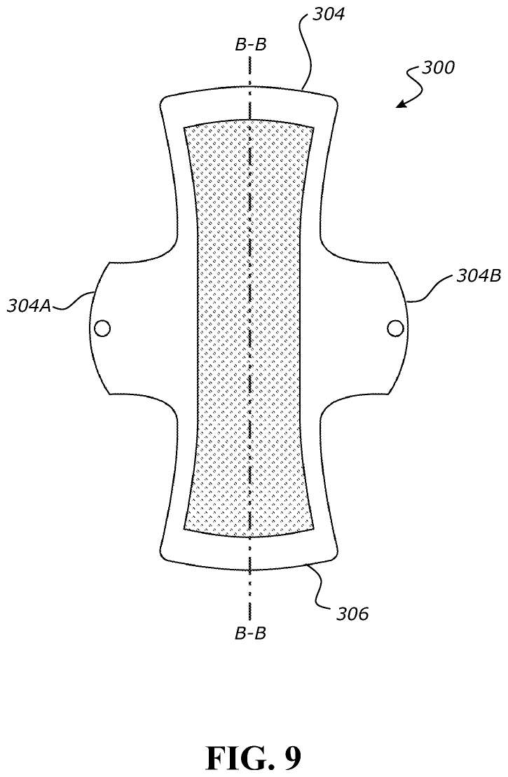

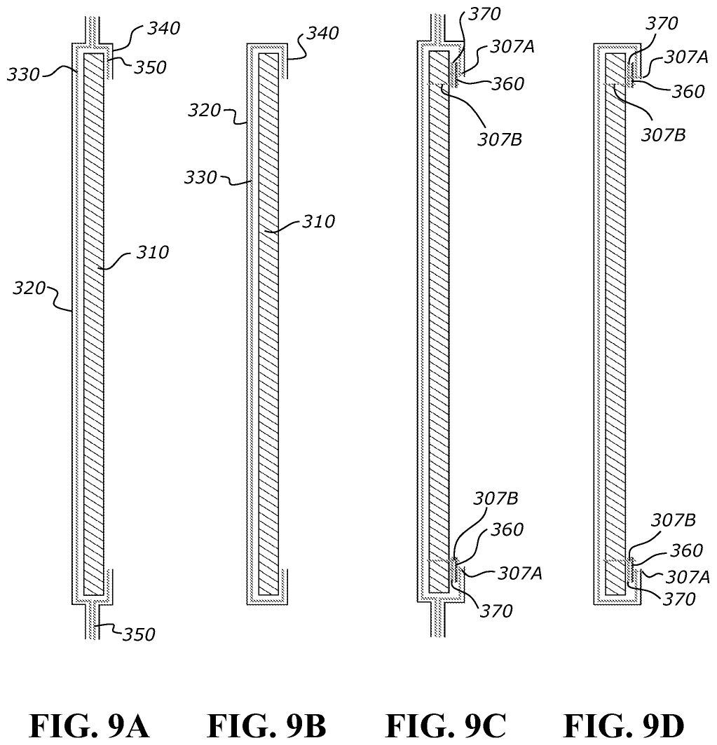

FIG. 8 shows an article 300 according to another preferred embodiment of the present invention. FIG. 9 shows B-B axis of the article of FIG. 8. FIGS. 8A-D are cut through views along A-A axis of FIG. 8 showing several different possible configurations of the different layers. FIGS. 9A-D are cut through views along B-B axis of FIG. 9 showing several different possible configurations of the different layers.

The article 300 may be used as a menstrual fluid absorbing and retaining article. The article 300 may also be suitable for absorbing and retaining any other body moisture.

The article 300 of this example is similar in most aspects to the article 100 described above and the differences can be identified by comparing the figures. In FIGS. 8, 8A-8D, 9 and 9A-9D, the features that are similar to those shown in figures relating to article 100 are identified with the same reference numeral, incremented by 200.

FIG. 8A is a cut through view of the article 300 showing one arrangement of the layers along axis A-A. Most of the description of the article 100 of a preferred embodiment above, equally applies to this embodiment of article 300 therefore need not be described again in too much detail. Only the main differences will be discussed. Similarly, the structure and orientations of various layers are self-evident from the cut through view of FIG. 8A and therefore need not be explained in detail.

As shown in FIG. 8A, portion of the adhesive layer 330 may be positioned or sandwiched between the insert 310 and the outer cover 320. In other words, adhesive layer 330 may be positioned or sandwiched between the insert 310 and the outer cover 320 except at and near the left and right sides 304A, 304B of the article 300 where insert 310 does not extend. Similarly, portion of the second adhesive layer 350 may be positioned or sandwiched between the insert 310 and the inner more layer 340. In other words, second adhesive layer 350 may be positioned or sandwiched between the insert 310 and the inner more layer 310 except at and near the left and right sides 304A, 304B of the article 300 where insert 310 does not extend. At or near the left and right sides 304A, 304B where the insert 310 does not extend, the adhesive layer 330 contacts the second adhesive layer 350.

In an alternative arrangement shown in FIG. 8B, the outer cover 330 may be formed or assembled to define an outer layer as well as the inner most layer of the article at or near the left and right sides 304A, 304B of the article 300. The adhesive layer 330 may be positioned or sandwiched between the insert 310 and the outer cover 320.

The alternative arrangement shown in FIG. 8C is substantially the same as that shown in FIG. 8A except it also shows a protection layer in the form of waterproof protective strips or frame 360 positioned between the second adhesive layer 350 and the surface of insert 310 that is configured to face towards the user's body. There may be a third adhesive layer 370 positioned between the protective strips or frame 360 and the insert 310 for bonding the protective strips or frame 360 with the insert. The bonding may be by application of heat bonding in addition to adhesive bonding. Preferably, the heat bonding is applied at temperature between 120 to 200 degrees Celsius. Use of third adhesive layer is optional as insert may be bonded to the protective strips or frame with the application of heat bonding. Although not shown, the articles 100, 200 as described above may also have such third adhesive layer for bonding together the protective strips or frame and the insert. As shown, there may be optional stitches 307A, 307B for extra durability. Stitch 307A may extend from the inner more layer 340 to the surface of insert 310 that is configured to face towards the user's body. Similarly, stitch 307B may extend from the protective strips or frame 360 to the surface of insert 310 that is configured to face away from the user's body. Use of these stitches are optional. Even with these optional stitches, the present invention still provides advantages because the number of stitches is very minimal and the bonding is still achieved mainly through the adhesives and by simply applying a compressive force or compression force together with heat on the article 300. Preferably, the heat bonding is applied at temperature between 120 to 200 degrees Celsius.

The alternative arrangement shown in FIG. 8D is substantially the same as that shown FIG. 8B except it also shows a protection layer in the form of waterproof protective strips or frame 360 positioned between the second adhesive layer 350 and the surface of insert 310 that is configured to face towards the user's body. There may be a third adhesive layer 370 positioned between the protective strips or frame 360 and the insert 310 for bonding the protective strips or frame 360 with the insert 310. The bonding may be by application of heat bonding in addition to adhesive bonding. Preferably, the heat bonding is applied at temperature between 120 to 200 degrees Celsius. Use of third adhesive layer 370 is optional as insert may be bonded to the protective strips or frame with the application of heat bonding. As shown, there may be optional stitches 307A, 307B for extra durability. Stitch 107A may extend from portion of the outer cover 320 that forms the inner most layer of the article to the surface of insert 310 that is configured to face towards the user's body. Similarly, stitch 307B may extend from the protective strips or frame 360 to the surface of insert 310 that is configured to face away from the user's body. Again, even with these optional stitches the advantages are still achieved for the same reasons as explained above.

FIGS. 9A-D are cut through views along B-B axis of FIG. 9 showing several different possible configurations of the different layers.

As shown in FIG. 9A, the outer cover 320 may be formed or assembled to define an outer layer of the article 300 at or near the top and bottom ends of the article 300. The adhesive layer 330 may be positioned or sandwiched between the insert 310 and the outer cover 320. The inner more layer 340 may be bonded to the insert 310 using second adhesive layer 350 positioned and sandwiched between the inner more layer and the insert. At or near the top and bottom ends 304, 306 of the article 300 where there is no insert, the outer cover 320 may be bonded to inner more layer with first adhesive layer 330 and the second adhesive layer 350.

In an alternative arrangement shown in FIG. 9B, the outer cover 320 may be formed or assembled to define an outer layer as well as the inner most layer of the article at or near the top end 304 and bottom end 306. The adhesive layer 330 may be positioned or sandwiched between the insert 310 and the outer cover 320.

The alternative arrangement shown in FIG. 9C is substantially the same as that shown FIG. 9A except that it also shows a protection layer in the form of waterproof protective strips or frame 360 positioned between the second adhesive layer 350 and the surface of insert 310 that is configured to face towards the user's body. There may be a third adhesive layer 370 positioned between the protective strips or frame 360 and the insert 310 for bonding the protective strips or frame 360 with the insert. The bonding may be by application of heat bonding in addition to adhesive bonding. Preferably, the heat bonding is applied at temperature between 120 to 200 degrees Celsius. Use of third adhesive layer 370 is optional as insert may be bonded to the protective strips or frame with the application of heat bonding. Although not shown, the articles 100, 200 as described above may also have such third adhesive layer for bonding together the protective strips or frame and the insert. Alternatively, such bonding may be achieved with heat bonding alone. As shown, there may be optional stitches 307A, 307B for extra durability. Stitch 307A may extend from the inner more layer 340 to the surface of insert 310 that is configured to face towards the user's body. Similarly, stitch 307B may extend from the protective strips or frame 360 to the surface of insert 310 that is configured to face away from the user's body. Use of these stitches are optional. Even with these optional stitches, the present invention still provides advantages for the reasons as already described above.

The alternative arrangement shown in FIG. 9D is substantially the same as that shown in FIG. 9B except that it also shows a protective layer in the form of waterproof protective strips or frame 360 positioned between the second adhesive layer 350 and the surface of insert 310 that is configured to face towards the user's body. There may be a third adhesive layer 370 positioned between the protective strips or frame 360 and the insert 310 for bonding the protective strips or frame 360 with the insert 310. As shown, there may be optional stitches 307A, 307B for extra durability. Stitch 107A may extend from portion of the outer cover 320 that forms the inner most layer of the article to the surface of insert 310 that is configured to face towards the user's body. Similarly, stitch 107B may extend from the protective strips or frame 360 to the surface of insert 310 that is configured to face away from the user's body. Again, even with these optional stitches the advantages are still achieved for the same reasons as explained above.

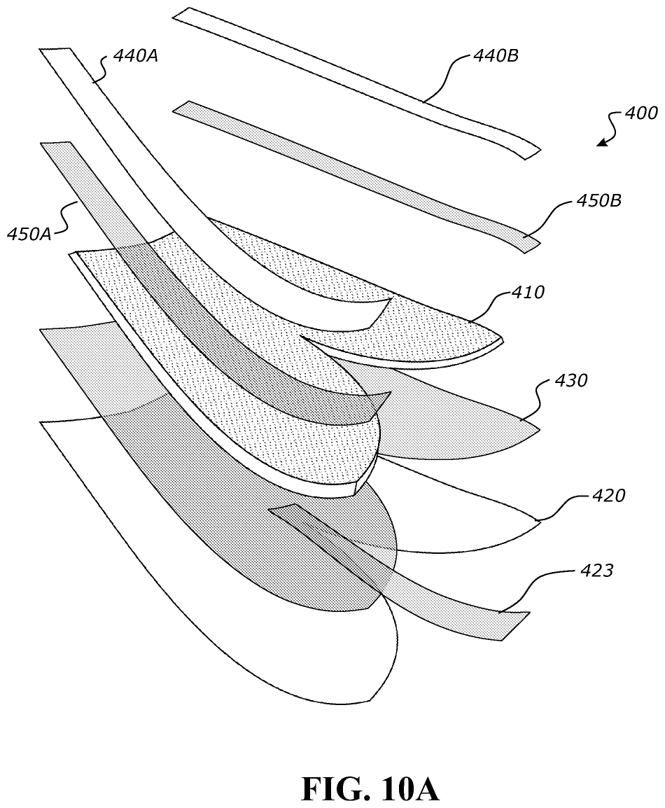

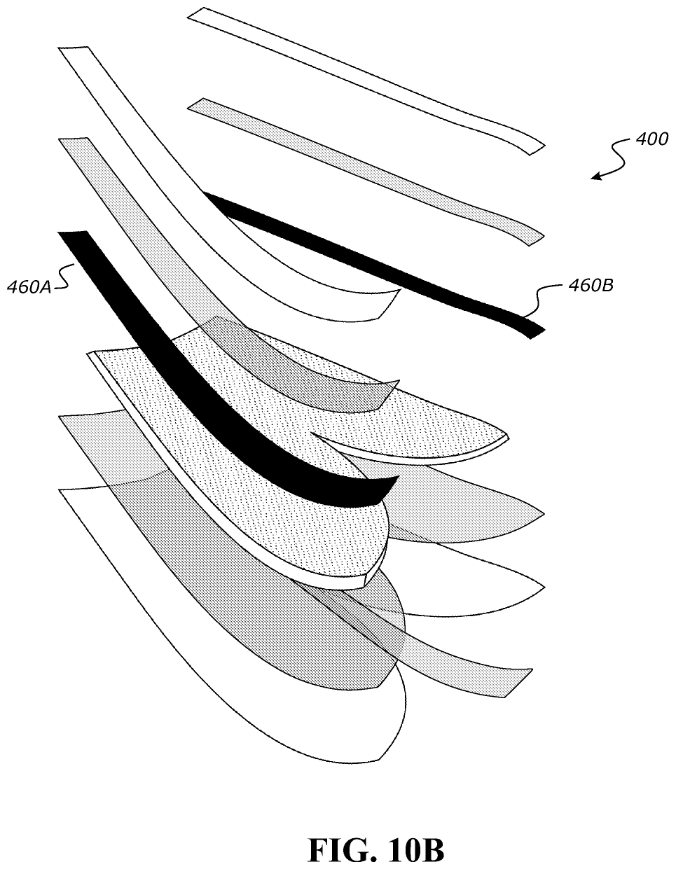

FIGS. 10A-10B show an exploded view of the reusable article 400 according to another embodiment of the present invention.

The article 400 is of or for use with an undergarment to be positioned adjacent a body of a user and to receive moisture from the body of the user. The user may be a person wearing the article. The article 400 may be used as a urine incontinence absorbing and retaining article (also referred to as a “male shield” or “male guard, “male pouch” or incontinence pouch”). The article 400 may also be suitable for absorbing and retaining any other moisture from the body of the user. Preferably, the article 400 is an incontinence article for female that is configured to be positioned adjacent a person's genital for receiving menstrual or other fluid from genital area of the person, including urine.

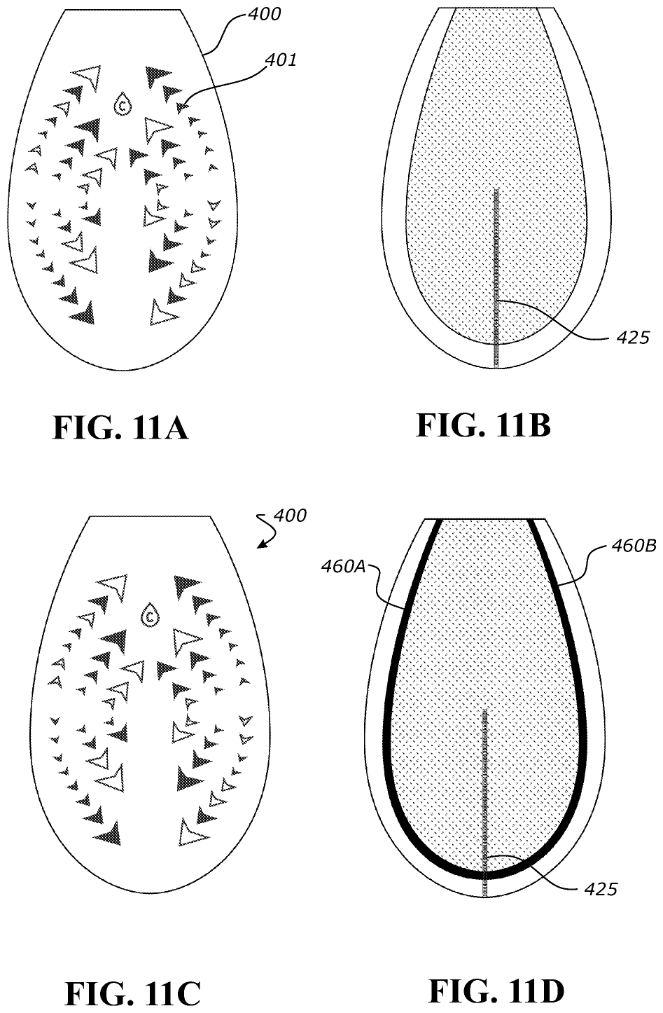

The article 400 of this example is similar in most aspects to the article 100 described above and the differences can be identified by comparing FIG. 1 with FIG. 10A and FIG. 3 with FIG. 10B, FIG. 2B with FIG. 11B, and FIG. 5A with FIG. 11D. In figures relating to the article 400, the features that are similar to those shown in figures relating to article 100 are identified with the same reference numeral, incremented by 300. Most of the description of the article 100 of a preferred embodiment above, equally applies to the article 300 and therefore need not be described again in too much detail. Only the main differences will be discussed.

One main difference is in the shape of the outer cover, the adhesive layer and the insert all of which are suitable shaped to function as the urine incontinence absorbing and retaining article.

The inner more layer are in the form of strips 440A, 440B that are located outwardly of the insert 410. A second adhesive layer 450 is also in the form of adhesive strips 450A, 450B. Adhesive strip 450A may be positioned or sandwiched between the insert 410 and the inner more layer strip 440A. Similarly, adhesive strip 450B may be positioned or sandwiched between the insert 410 and the inner more layer strip 440B. Therefore, the adhesive strips 450A, 450B may interconnect the insert 420 and the inner more strips 440A, 440B.

The inner more strips 440A, 440B may be made from the same textiles as the outer cover 420. Inner more strips 440A, 440B and the outer cover 420 may be made from woven or knitted textiles. Any suitable woven or knitted textiles may be used. The outer cover 420 and the inner more strips 440A, 440B may be moisture resistant. Preferably, a moisture resistant solution is applied to the outer cover 420 and the inner more strips 440A, 440B. The outer cover 420 and the inner more strips 440A, 440B may be coated with a moisture resistant solution.

The inner more strips 440A, 440B and the adhesive strips 450A, 450B may longitudinally extend along the side edges 410C, 410D of the insert 410 in a direction that is from a top end 404 of the article to the bottom end 406 of the article 400 or vice versa. More specifically, the inner more strip 440A and the adhesive strip 450A may longitudinally extend along the side edge 410C of the insert 410 in a direction that is from a top end of the article to the bottom end of the article 400 or vice versa. Similarly, the inner more strip 440B and the adhesive strip 450B may longitudinally extend along the side edge of the insert 410 in a direction that is from a top end of the article to the bottom end of the article 400 or vice versa. The side edges may longitudinally extend between the first and second edges.

In some embodiment the outer cover 420 may already come with stitches 425 that are provided to allow the outer cover 420 to be of a particular shape. In such case, a stitch bonding adhesive layer 423 may optionally be provided to extend along and conceal such stitches for extra protection. The stitch bonding adhesive layer 423 may be a stitch binding glue seal and may be non-woven. The stitch bonding adhesive layer 423 may be made from the same material as the adhesive layer 420. It is to be noted that the stitches 425 may not be present in the outer cover 420 and alternative methods to stitches (e.g., creases). may be used to create the shape of the article 400 but even if present they are pre-formed stitches and the stitches that is configured to bond multiple layers together. The multiple layers are still bonded by applying compressive force on the article 400 and using the adhesives, preferably adhesive and heat bonding. Preferably, the heat bonding is applied at temperature between 120 to 200 degrees Celsius.

Optional waterproof protective layer which in this example is in the form of protective strips 460A, 460B may also be present as shown in FIG. 10B. These are similar to protective strips 160A, 160B as described above both in terms of their construction and orientation and therefore need not be described again. Such protective strips 460A, 460B helps avoid the transfer of moisture from the layers to outer textiles from the sides and bottom of the article.

Other than the above mentioned differences and the differences that are obvious from the figures, all other features of the article 400 including the properties of its various layers may be same as that described above with reference to article 100.

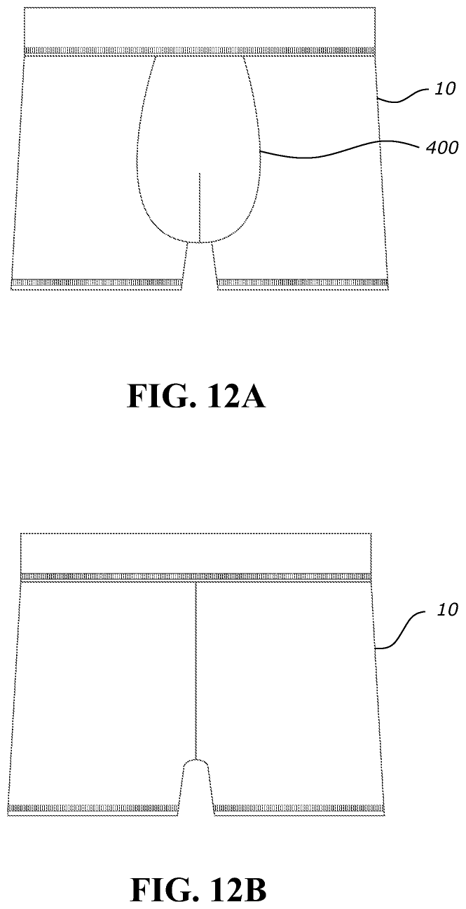

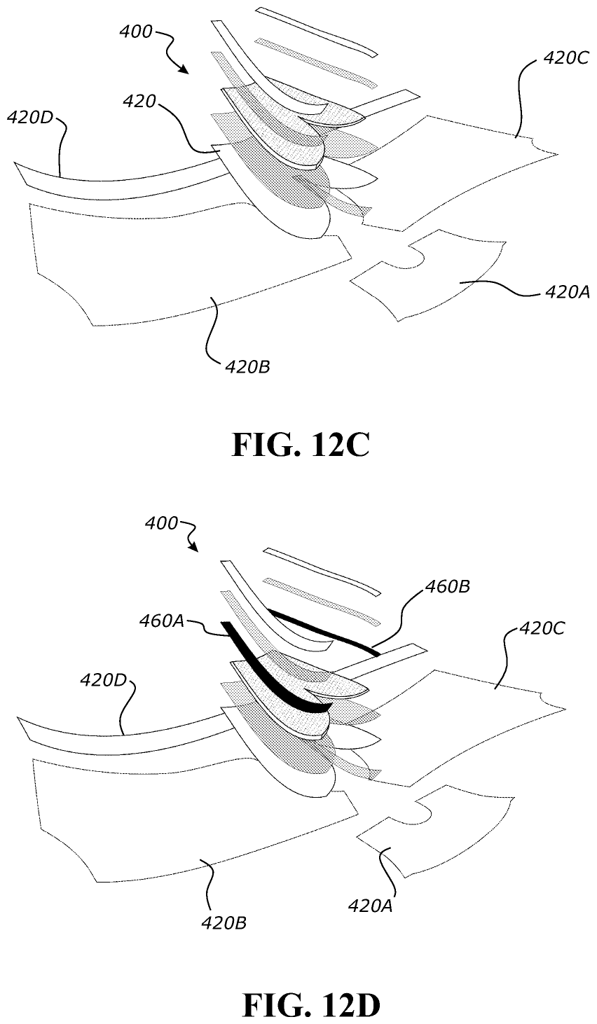

FIGS. 12A-B are front and rear views of a men's/male's underwear 10 comprising the article according to a further embodiment of the present invention that is same or similar to the article of FIG. 10A or 10B. Hence, same reference numeral has been used for this embodiment. When applied to the underwear, protective strips may not be present, but most preferably it is present for the technical advantages as already described above.

FIG. 12C shows an exploded view showing the article 400 forming part of male underwear. FIG. 12D shows an exploded view showing the article 400 forming part of male underwear with optional protective layer which in this example is in the form of waterproof protective strips 460A, 460B. The outer cover 420 may be part of a male underwear panel and may be made from knitted textiles and are preferably highly stretchable for comfort. The other panels 420A, 420B, 420C of the underwear are also shown. These panels may also be made from knitted textiles and are preferably highly stretchable. A waist elastic 420D of the underwear is also shown. These parts of underwear may be pre-coupled with the outer cover 420 using any suitable method either prior or after the bonding of the various layers of the article 400 using compressive force. Preferably, heat bonding is applied at temperature between 120 to 200 degrees Celsius.

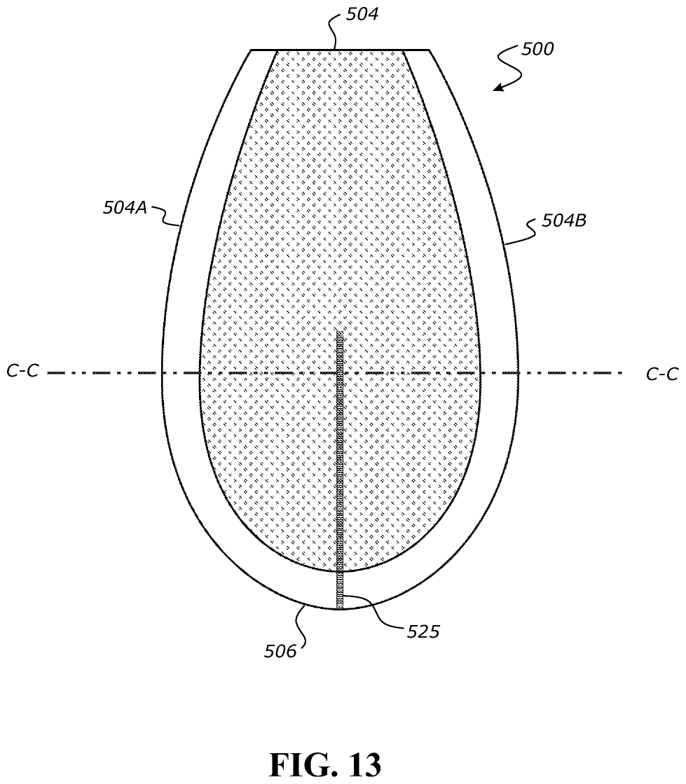

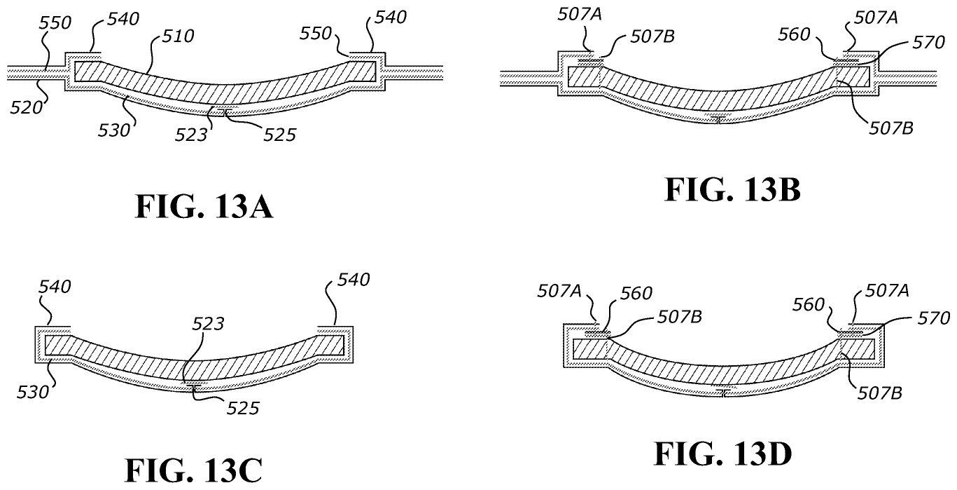

FIG. 13 shows an article 500 according to another preferred embodiment of the present invention. The article 500 may be used as a urine incontinence absorbing and retaining article. The article 500 may also be suitable for absorbing and retaining any other moisture from the body of the user.

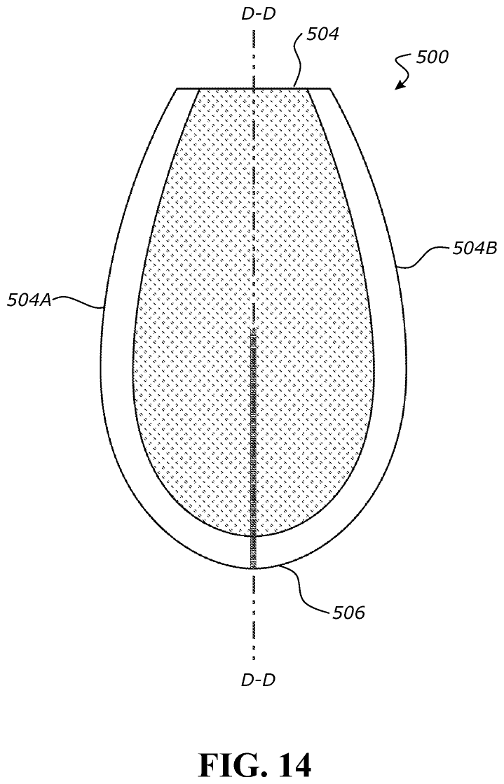

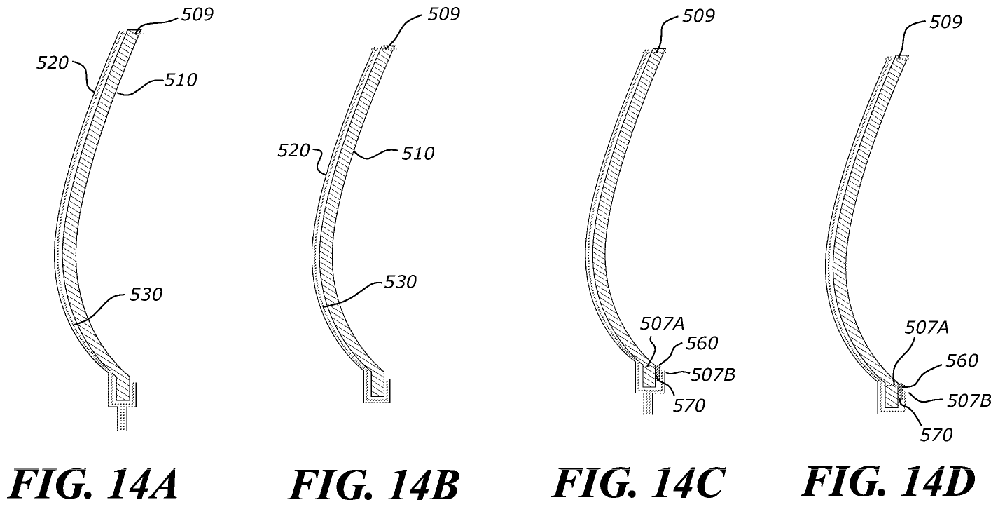

The article 500 of this example is similar in most aspects to the article 400 described above. FIG. 14 shows C-C axis of the article of FIG. 13. FIGS. 13A-D are cut through views along C-AC axis of FIG. 13 showing several different possible configurations of the different layers. FIGS. 14A-D are cut through views along A-D axis of FIG. 14 showing several different possible configurations of the different layers.

The article 500 of this example is similar in most aspects to the article 400 described above and the differences can be identified by comparing the figures. In FIGS. 13, 13A-D, 14 and 14A-D, the features that are similar to those shown in figures relating to article 400 are identified with the same reference numeral, incremented by 100.

FIG. 13A is a cut through view of the article 500 showing one arrangement of the layers along axis C-C. Most of the description of the article 100 of a preferred embodiment above, equally applies to this embodiment of article 500 therefore need not be described again in too much detail. Only the main differences will be discussed. Similarly, the structure and orientations of various layers are self-evident from the cut through view of FIG. 13A and therefore need not be explained in detail.

As shown in FIG. 13A, portion of the adhesive layer 530 may be positioned or sandwiched between the insert 510 and the outer cover 520. In other words, adhesive layer 530 may be positioned or sandwiched between the insert 510 and the outer cover 520 except at and near the left and right sides 504A, 504B of the article 500 where insert 510 does not extend. Similarly, portion of the second adhesive layer 550 may be positioned or sandwiched between the insert 510 and the inner more layer 540. In other words, second adhesive layer 550 may be positioned or sandwiched between the insert 510 and the inner more layer 510 except at and near the left and right sides 504A, 504B of the article 500 where insert 510 does not extend. At or near the left and right sides 504A, 504B of the article 500 where insert 510 does not extend, the adhesive layer 530 contacts the second adhesive layer. As shown outer cover 220 may already come with stitches 525 that are provided to allow the outer cover 520 to be of a particular shape. In such case, a stitch bonding adhesive layer 523 may be used between the adhesive layer 530 and insert 510 as shown in the drawing to cover the stitches 523.

The alternative arrangement shown in FIG. 13B is substantially the same as the arrangement shown in FIG. 13A except it also shows a waterproof protective strips or frame 560 positioned between the second adhesive layer 550 and the surface of insert 510 that is configured to face towards the user's body. There may be a third adhesive layer 570 positioned between the protective strips or frame 360 and the insert 310 for bonding the protective strips or frame 360 with the insert. Although not shown, the article 400 as described above may also have such third adhesive layer for bonding together the protective strips or frame and the insert. As shown, there may be optional stitches 507A, 507B for extra durability. Stitch 507A may extend from the inner more layer 440 to the surface of insert 510 that is configured to face towards the user's body. Similarly, stitch 507B may extend from the protective strips or frame 560 to the surface of insert 510 that is configured to face away from the user's body. Use of these stitches are optional. Even with these optional stitches, the present invention still provides advantages because the number of stitches is very minimal, and the main bonding is still achieved purely through adhesives (using adhesive bonding or adhesive and heat bonding) and by simply applying a compressive force on the article 500. Preferably, the heat bonding is applied at temperature between 120 to 200 degrees Celsius.

As shown in the alternative arrangement of FIG. 13C, the outer cover 520 may be formed or assembled to define an outer layer as well as the inner most layer of the article at or near the left and right sides. The adhesive layer 530 may be positioned or sandwiched between the insert 510 and the outer cover 520. As shown outer cover 520 may already come with stitches 525 that are provided to allow the outer cover 420 to be of a particular shape. In such case, a stitch bonding adhesive layer 523 may be used between the adhesive layer 530 and adsorbent insert 510 as shown in the drawing to cover the stitches 523.

The alternative arrangement shown in FIG. 13D is substantially the same as the arrangement shown in FIG. 13C except it also shows a waterproof protective strips or frame 560 positioned between the second adhesive layer 550 and the surface of insert 510 that is configured to face towards the user's body. There may be a third adhesive layer 570 positioned between the protective strips or frame 560 and the insert 510 for bonding the protective strips or frame 560 with the insert. As shown, there may be optional stitches 507A, 507B for extra durability. Stitch 507A may extend from portion of the outer cover 520 that forms the inner most layer of the article to the surface of insert 510 that is configured to face towards the user's body. Similarly, stitch 507B may extend from the protective strips or frame 560 to the surface of insert 510 that is configured to face away from the user's body. Again, even with these optional stitches the advantages are still achieved for the same reasons as explained above.

FIG. 14D is one embodiment of a cut through view of article 500 along axis D-D. Here the portion of the adhesive layer 530 may be positioned or sandwiched between the insert 510 and the outer cover 520. In other words, adhesive layer 530 may be positioned or sandwiched between the insert 510 and the outer cover 520 except at or near the bottom end 506 of the article 500 where insert 510 does not extend. Similarly, portion of the second adhesive layer 550 may be positioned or sandwiched between the insert 510 and the inner more layer 540. In other words, second adhesive layer 550 may be positioned or sandwiched between the insert 510 and the inner more layer 510 except at and near bottom end 506 of the article 500 where insert 510 does not extend. At or near the bottom end 506 where the insert 510 does not extend, the adhesive layer 530 contacts the second adhesive layer 550. An optional stitch 209A may appear at or near the top end 504 of the article. But, even with such optional stitches 504, the present invention still provides advantages because the number of stitches are very minimal and the main bonding is still achieved purely through adhesives (preferably using adhesive or adhesive and heat bonding) and by simply applying a compressive force on the article 500. Preferably, the heat bonding is applied at the temperature between 120 degrees Celsius and 200 degrees Celsius.

Other than the above mentioned differences and the differences that are obvious from the figures, all other features of the article 500 including the properties of its various layers may be same as that described above with reference to article 100.

FIG. 14B is a cut through view of the article 500 showing one arrangement of the layers along axis D-D of FIG. 14. As shown, the outer cover 520 may be formed or assembled to define an outer layer as well as the inner most layer of the article at or near bottom end 506. The adhesive layer 530 may be positioned or sandwiched between the insert 510 and the outer cover 520. Optional stitches 509 may be present at or near the top end 504 for extra durability but that is not essential.

The alternative arrangement of FIG. 14C is substantially the same as that of FIG. 14A except that it also shows protective layer in the form of a waterproof protective strips or frame 560 positioned between the second adhesive layer 550 and the surface of insert 510 that is configured to face towards the user's body. There may be a third adhesive layer 570 positioned between the protective strips or frame 560 and the insert 510 for bonding the protective strips or frame 560 with the insert 510. Although not shown, the article 400 as described above may also have such third adhesive layer for bonding together the protective strips or frame and the insert. Use of third adhesive layer is optional and bonding may be achieved by head bonding (preferably applied at the temperature between 120 to 200 degrees Celsius). As shown, there may be optional stitches 507A, 507B for extra durability. Stitch 507A may extend from the inner more layer 540 to the surface of insert 510 that is configured to face towards the user's body. Similarly, stitch 507B may extend from the protective strips or frame 560 to the surface of insert 510 that is configured to face away from the user's body. Use of these stitches are optional. Even with these optional stitches, the present invention still provides advantages as already explained above.

The alternative arrangement of FIG. 14D is substantially the same as that of FIG. 14B except that it also shows a waterproof protective strips or frame 560 positioned between the second adhesive layer 550 and the surface of insert 510 that is configured to face towards the user's body. There may be a third adhesive layer 570 positioned between the protective strips or frame 560 and the insert 510 for bonding the protective strips or frame 560 with the insert. Use of third adhesive layer is optional and bonding may be achieved by head bonding (preferably applied at the temperature between 120 to 200 degrees Celsius). As shown, there may be optional stitches 507A, 507B for extra durability. Stitch 507A may extend from portion of the outer cover 520 that forms the inner most layer of the article to the surface of insert 510 that is configured to face towards the user's body. Similarly, stitch 507B may extend from the protective strips or frame 560 to the surface of insert 510 that is configured to face away from the user's body. Again, even with these optional stitches the advantages are still achieved for the same reasons as explained above.

It will of course be realised that while the foregoing has been given by way of illustrative example of the present invention, all such modifications and variations thereto as would be apparent to a person skilled in the art are deemed to fall within the broad scope and ambit of the various aspects of invention as defined in the claims.

From the above, it can be appreciated that the present invention allows layers of the article to be bonded together by simply arranging the layers as described above applying a compressive force on the article. This eliminates or at least reduces stitching and/or other complex process for bonding the layers together thereby significantly reducing the manufacturing process, costs and time. Also, complex machinery will note be required for manufacturing the article 100. Also, no or less stitching means the article is less prone to leakage. Preferably, the article is leakproof.

One or more non-limiting advantages of the present invention may be as follows:
Simpler design
Cost effective
Easy to assemble
No complex machinery is required for assembly of layers as the layers can be adhesively bonded simply by applying compressive force on the article—i.e., pressing and preferably also applying heat.
Reduction in manufacturing time
Less labour intensive to manufacture/assemble as no complex bonding process
Reusable and plastic free article-Environmentally friendly
Free of any harmful or hazardous chemicals
Can be washed and tumbled dried.
Less prone to leakages due to less or no stitching
Absorbency speed, levels & liquid retention abilities are not compromised whist significantly reducing the number of steps, time, and complexity of the assembly of layers during manufacturing

1. A method of constructing a reusable moisture retaining article of or for use with an undergarment to be positioned adjacent a body of a user and to absorb and retain moisture that is a body moisture from the user, the method comprising:
providing a reusable moisture absorbing and retaining insert at least a portion of which is exposed or configured to be exposed to define an inner layer of the article, the inner layer being configured to be located adjacent the user's body and facing towards the user's body for being in bodily contact with the user to directly absorb and retain the moisture from the user;
providing an outer cover that is configured to define an outer layer of the article;
positioning at least a portion of an adhesive layer that is a first adhesive layer between the insert and the outer cover, wherein the first adhesive layer extends along at least a part of a surface area of the insert and the outer cover for interconnecting the insert and the outer cover;
applying a compressive force to at least the insert and the outer cover; and
applying heat and adhesive bonding to bond the insert and the outer cover together using the first adhesive layer, wherein the heat bonding is applied at a temperature range correlating to a melting point of the insert and the outer cover thereby allowing the insert and outer cover to bond together;
the method further comprising:
providing an inner more layer;
providing a protective layer that is waterproof;
providing a further adhesive layer that is a second adhesive layer;
positioning the protective layer on a surface of the insert that is configured to face towards the user's body;
positioning the second adhesive layer so that at least a portion of the second adhesive layer is located between the protective layer and the inner more layer for interconnecting at least the protective layer and the inner more layer; and
applying a further compressive force to at least the insert and the at least one inner more layer; and
applying a heat and adhesive bonding thereby bonding the protective layer with the inner more layer and the insert.

2. A method as claimed in claim 1, wherein the method comprises positioning at least a further portion of the second adhesive layer between the insert and the inner more layer for interconnecting the insert and the inner more layer thereby bonding the insert with inner more layer using the second adhesive layer.

3. A method as claimed in claim 1, wherein the method comprises positioning each layer of the article so that they are substantially parallel to each other.

4. A method as claimed in claim 1, wherein the adhesive layer is coextensive with the insert and/or the outer cover.

5-11. (canceled)

12. A method as claimed in claim 1, wherein at least one of the outer cover and the inner more layer is coextensive with the insert.

13. A method as claimed in claim 1, wherein at least one of the outer cover and the inner more layer is moisture resistant, a moisture resistant solution is applied to the outer cover, and/or the outer cover is coated with a moisture resistant solution.

14. (canceled)

15. A method as claimed in claim 1, wherein the second adhesive layer is at least one adhesive strip.

16. (canceled)

17. A method as claimed in claim 1, wherein the inner more layer comprises or is in the form of a first inner more strip and a second inner more strip and the second adhesive layer comprises or is in the form of a first adhesive strip and a second adhesive strip, wherein the method comprises: locating the first inner more strip outwardly of the insert and bonding the first inner more strip to the insert and the protective layer using the first adhesive strip; and locating the second inner more strip outwardly of the insert and bonding the second inner more strip to the insert and the protective layer using the second adhesive strip, wherein the method further comprises positioning or sandwiching at least a portion of the first adhesive strip between the insert and the first inner more strip thereby bonding the first inner more strip and the insert, and positioning or sandwiching at least a portion of the second adhesive strip between the insert and the second inner more strip thereby bonding the second inner more strip and the insert.

18. A method as claimed in claim 1, wherein the inner more layer defines a window to allow the portion of insert to be exposed or configured to be exposed.

19. A method as claimed in claim 18, wherein the method comprises exposing all of the insert from the window to form the inner layer of the article save for its edges.

20. A method as claimed in claim 1, wherein the protective layer is at least one protective strip and the method comprises positioning or sandwiching the at least one protective strip between the insert and the second adhesive layer on the surface of insert that is configured to face towards the user's body.

21. A method as claimed in claim 1, wherein the protective layer is at least two protective strips and the method comprises positioning or sandwiching the at least two protective strips between the insert and the second adhesive layer on the surface of insert that is configured to face towards the user's body and along at least two edges of the insert.

22. A method as claimed claim 1, wherein the method comprises positioning or sandwiching the protective strip(s) between the insert and the second adhesive layer on the surface of the insert that is configured to face towards the user's body and along all edges/perimeter of the insert.

23. A method as claimed in claim 1, wherein the method comprises forming the protective layer as a single piece frame.

24. (canceled)

25. A reusable moisture retaining article of or for use with an undergarment to be positioned adjacent a body of a user and to absorb and retain moisture from the body of the user, the article being a reusable article and comprising:
a reusable moisture absorbing and retaining insert at least a portion of which is exposed or configured to be exposed to define an inner layer of the article, the inner layer being configured to be located adjacent the user's body and facing towards the user's body for being in bodily contact with the user to directly absorb and retain the moisture from the body of the user;
an outer cover that is formed or assembled to define an outer layer of the article; and
an adhesive layer that is a first adhesive layer, at least a portion of the adhesive layer being positioned or sandwiched between a surface of the insert that is configured to face towards the outer cover and a surface of the outer cover that is configured to face towards the insert thereby interconnecting the insert and the outer cover, the insert and the outer cover are bonded together by applying a compressive force to at least the insert and the outer cover and by applying heat and adhesive bonding to bond the insert and outer cover together using the first adhesive layer, wherein the heat bonding is applied at a temperature range correlating to a melting point of the insert and the outer cover thereby allowing the insert and outer cover to bond together;
the article further comprising:
a protective layer that is waterproof;
an adhesive layer that is a second adhesive layer; and
an inner more layer;
wherein the protective layer is positioned on a surface of the insert that is configured to face towards the user's body, at least a portion of the second adhesive layer is located between the protective layer and the inner more layer interconnecting at least the protective layer and the inner more layer, and the protective layer is bonded with the inner more layer and the insert by adhesive and heat bonding.

26. An article as claimed in claim 25, wherein a further portion of the second adhesive layer is located between the insert and the inner more layer for interconnecting and bonding the insert and the inner more layer.

27-36. (canceled)

37. An article as claimed in claim 25, wherein the outer cover is a leakproof layer.

38-45. (canceled)

46. An article as claimed in claim 25, wherein the inner more layer defines a window to allow the portion of insert to be exposed or configured to be exposed.

47. An article as claimed in claim 25, wherein all of the-insert is exposed or configured to be exposed to form the inner layer of the article save for its edges.

48-52. (canceled)

53. An article as claimed in claim 25, wherein the inner more layer and the second adhesive layer only cover the perimeter of the insert on its surface that is configured to face towards the user's body.

54-61. (canceled)
